(19) United States
(12) Patent Application Publication
(10) Pub. No.: US 2012/0210459 A1
(43) Pub. Date: Aug. 16, 2012

(54) Design and Implementation of Novel and/or Enhanced Bacterial Microcompartments for Customizing Metabolism

(75) Inventors: Cheryl A. Kerfeld, Walnut Creek, CA (US); Dominique Loque, Albany, CA (US)

(73) Assignee: THE REGENTS OF THE UNIVERSITY OF CALIFORNIA, Oakland, CA (US)

(21) Appl. No.: 13/367,260

(22) Filed: Feb. 6, 2012

Related U.S. Application Data
(63) Continuation-in-part of application No. PCT/US2010/044455, filed on Aug. 4, 2010.
(60) Provisional application No. 61/231,246, filed on Aug. 4, 2009.

(51) Int. Cl. C12N 15/82; C12N 1/15; A01H 5/10; C40B 40/08; C07H 21/04; A01H 5/00; C12N 1/21; C12N 1/19

(52) U.S. Cl. 800/278; 435/252.3; 435/254.11; 435/254.2; 435/320.1; 506/17; 536/23.7; 800/298

(57) ABSTRACT

Herein is described a bacterial microcompartment catalog comprising a total of 634 gene sequences encoding bacterial microcompartments, the proteins of each can be inserted into a host organism and if needed, expressed using an inducible expression system. Disclosed are at least 32 types of gene clusters which provide microcompartments having metabolizing or other enzyme activity. The expression of these microcompartments can be used to provide or enhance an organism's carbon fixation and/or sequestration activity or biomass production or, generally speaking additional or enhanced metabolic activities to an organism.

### CROSS-REFERENCE TO RELATED APPLICATIONS

This application is a continuation-in-part of International Application No. PCT/US2010/44455 filed on Aug. 4, 2010, which claims priority to U.S. Provisional Patent Application No. 61/231,246 filed on Aug. 4, 2009, both of which are hereby incorporated by reference in their entirety.

### STATEMENT OF GOVERNMENTAL SUPPORT

This invention was made with government support under Contract No. DE-AC02-05CH11231 awarded by U.S. Department of Energy. The government has certain rights in this invention.

### REFERENCE TO SEQUENCE LISTING AND TABLES

The attached sequence listing is hereby incorporated by reference.

The attached Table 2 is hereby incorporated by reference.

### BACKGROUND OF THE INVENTION

1. Field of the Invention

The present invention relates to method for designing and implementing novel and/or enhanced bacterial microcompartments for customizing metabolism in various organisms such as bacteria, archaea, plants, algae, and other eukaryotes through genome modification. The present invention also relates to modified organisms having enhanced biomass production and CO₂ sequestration abilities.

2. Related Art

Bacterial microcompartments are primitive protein-based organelles that sequester specific metabolic pathways in bacterial cells. The prototypical bacterial microcompartment is the carboxysome, a bacterial polyhedral organelle which increases the efficiency of CO₂ fixation by encapsulating RuBisCO and carbonic anhydrase and other proteins. They can be divided into two types: alpha-type carboxysomes and beta-type carboxysomes (FIGS. 13, 25, 26).

For many years carboxysomes were the only known polyhedral microcompartments known in bacteria. Subsequently, homologues of carboxysome shell proteins were reported in Salmonella enterica serovar Typhimurium, where they constitute part of a cluster of genes involved in the coenzyme B₁₂-dependent metabolism of 1,2-propanediol (Pdu bacterial micrompartment) and in a second gene cluster, constituting a bacterial microcompartment for the metabolism of ethanolamine. More recently we have bioinformatically extended the observations of the potential to form bacterial microcompartments in diverse species of bacteria; however for many of these the predicted function has yet to be experimentally verified.

There has been recent interest in using microorganisms and algae in the production and processing of biofuels.

### BRIEF SUMMARY OF THE INVENTION

The present invention provides method for designing and implementing novel and/or enhanced bacterial microcompartments for customizing metabolism in various organisms such as plants, algae, bacteria, and eukaryotes. It was found that genes with homology to the conserved bacterial microcompartment domains Pfam00936 and/or Pfam03319 along with any other genes that are associated, co-regulated or identifiable as in a gene cluster with these Pfam00936 and/or Pfam03319 homologs, can be inserted into the genome of another organism, thereby providing enhanced or new activity to the transformed organism.

Various compositions comprising nucleotide and/or amino acid sequences comprising bacterial microcompartments are herein described. Specifically, the present invention provides microcompartment nucleic acids and polypeptides having a sequence set forth in SEQ ID NOs: 1-1268 and variants, homologs and fragments thereof. The present invention further provides compositions and methods directed to enhancing or customizing metabolism in various organisms.

In one aspect of the invention, an isolated nucleic acid molecule is inserted into a genome of an organism such as a plant, algae, bacteria or eukaryote, wherein the nucleic acid molecule encodes a protein or RNA molecule encoding bacterial microcompartment proteins not naturally present in the organism, thus providing enhanced or new activity. In one embodiment, the present methods and sequences provide these organisms with microcompartments that provide enhanced biomass production and CO₂ sequestration/fixation abilities.

In one embodiment, the bacterial microcompartment genes or their homologs are isolated from bacteria and clusters of which are grouped into 32 Groups and subgroups and shown in Table 1. Proxy organisms for each Group found in Table 1. In another aspect, an isolated nucleic acid, wherein the sequence is selected from the group consisting of odd-numbered sequences from SEQ ID NOS:1-1268.

In another aspect, the encoded protein or RNA molecule having biomass production and CO₂ sequestration or carbon fixation activity. In one embodiment, a microcompartment protein expressed in vitro from an isolated gene or RNA molecule and selected from the odd numbered sequences from SEQ ID NOS: 1-1268. In another embodiment, the isolated protein having carbon fixation activity, comprising a sequence selected from even-numbered sequences from SEQ ID NOS: 1-1268.

The isolated protein or RNA molecule having carbon fixation activity, wherein the protein or RNA molecule or homologs having the potential for bacterial microcompartment formation is isolated from organisms such as those in Table 1. In other embodiments, a cluster or group of proteins or RNA molecule or homologs having the potential for bacterial microcompartment formation is isolated from organisms such as the Groups as defined in Table 3 or any organisms' bacterial microcompartment gene clusters which can be defined as collections of genes that encode Pfam00936 and or Pfam03319 and genes in proximity to or co-regulated with expression of genes encoding Pfam00936 and or Pfam03319.

In another aspect, the nucleic acid molecule encoding microcompartment expression products, and isolated according to the prescribed method for inserting microcompartment genes in a genome, wherein said nucleotide sequence is optimized for expression in the host organism. An expression cassette comprising the nucleotide sequence operably linked to a promoter that drives expression in the host organism. The expression cassette further comprising an operably linked polynucleotide encoding a signal peptide if required.

In another embodiment, the nucleic acid molecule comprising a cluster of bacterial microcompartment genes, wherein the cluster comprising more than one bacterial compartment gene. The cluster of genes containing one or more occurrences of Pfam00936 and/or Pfam03319 wherein all contiguous genes are not greater than about 300 bp from one another or are distal in the genome (including in plasmids), but co-regulated/expressed with bacterial microcompartment genes. Thus, in one embodiment, an expression cassette comprising a nucleic acid molecule comprising a cluster of bacterial compartment genes.

In another aspect, a plant comprising in its genome at least one stably incorporated expression cassette, said expression cassette comprising a heterologous nucleotide sequence encoding a bacterial microcompartment operably linked to a promoter that drives expression in the plant, wherein the plant displays increased carbon fixation activity. The promoter is preferably an inducible promoter. In another embodiment, a transformed seed of the plant displaying increased carbon fixation activity.

In another aspect, a cell comprising in its genome at least one stably incorporated expression cassette, said expression cassette comprising a heterologous nucleotide sequence isolated according to the method of identifying microcompartment genes from a genome, operably linked to a promoter that drives expression in the cell.

In another aspect, a method for enhancing inorganic carbon fixation in a photosynthetic organism, said method comprising introducing into a photosynthetic organism at least one expression cassette, said expression cassette comprising a heterologous nucleotide sequence encoding a bacterial microcompartment and operably linked to a promoter that drives expression in the photosynthetic organism. In one embodiment, an expression cassette comprising a nucleotide sequence encoding a bacterial microcompartment sequence and operably linked to a promoter that drives expression in algae. In another embodiment, transformed photosynthetic microorganism comprising at least one expression cassette.

According to still further features in the described preferred embodiments the genetic transformation is effected by a method selected from the group consisting of Agrobaterium mediated transformation, plasmid-mediated transformation, electroporation, uptake via natural competence and particle bombardment.

According to still further features in the described preferred embodiments the transformation is effected by a method selected from the group consisting of plasmid-mediated transformation, natural competence for nucleic acid uptake, viral transformation, electroporation and particle bombardment.

### BRIEF DESCRIPTION OF THE DRAWINGS

FIG. 1 shows the various Groups of gene clusters, their function if known and lists a proxy organism in which this gene cluster is found.

FIGS. 2A-26A and also 13C show the legend and assign a color and shape for each enzyme or protein that comprises or has activity within a compartment in the Group proxy organism.

FIGS. 2B, 3B, 4B, etc. to 20B and also 13D show the Group microcompartment cluster as observed in various other organisms.

FIG. 2A shows the microcompartment gene cluster found in Group 1 proxy organism, Mycobacterium smegmatis str. MC2 155. FIG. 2B shows the Group 1 microcompartment also is present on other organisms.

FIG. 3A shows the microcompartment gene cluster found in Group 2 proxy organism, Ruminococcus obeum ATCC 29174. FIG. 3B shows the Group 2 microcompartment also is present on other organisms.

FIG. 4A shows the microcompartment gene cluster found in Group 3 proxy organism, Alkaliphilus metalliredigens QYMF. FIG. 4B shows the Group 3 microcompartment also is present on other organisms.

FIG. 5A shows the microcompartment gene cluster found in Group 4 proxy organism, E. coli CFT073. FIG. 5B shows the Group 4 microcompartment also is present on other organisms.

FIG. 6A shows the microcompartment gene cluster found in Group 5 proxy organism, Rhodopseudomonas palustris BisB18. FIG. 6B shows the Group 5 microcompartment also is present on other organisms.

FIG. 7A shows the microcompartment gene cluster found in Group 6 proxy organism, Shewanella putrefaciens CN-32. FIG. 7B shows the Group 6 microcompartment also is present on other organisms.

FIG. 8A shows the microcompartment gene cluster found in Group 7 proxy organism, E. coli UTI89. FIG. 8B shows the Group 7 microcompartment also is present on other organisms.

FIG. 9A shows the microcompartment gene cluster found in Group 8 proxy organism, Desulfatibacillum alkenivorans AK-01. FIG. 9B shows the Group 8 microcompartment also is present on other organisms.

FIG. 10A shows the microcompartment gene cluster found in Group 9 proxy organism, Blastopirellula marina DSM 3645. FIG. 10B shows the Group 9 microcompartment also is present on other organisms.

FIG. 11A shows the microcompartment gene cluster found in Group 10 proxy organism, Methylibium petroleiphilum. FIG. 11B shows the Group 10 microcompartment also is present on other organisms.

FIG. 12A shows the microcompartment gene cluster found in Group 11 proxy organism, Haliangium ochraceum SMP-2. FIG. 12B shows the Group 11 microcompartment also is present on other organisms.

FIG. 13A shows the microcompartment gene cluster found in Group 12 proxy organism, Anabaena variabalis. FIG. 13B shows the Group 12 microcompartment also is present on other organisms. FIG. 13C shows the microcompartment gene cluster found in Group 12A proxy organism, Trichodesmium erythraeum. FIG. 13D shows the Group 12A microcompartment also is present on other organisms.

FIG. 14A shows the microcompartment gene cluster found in Group 13 proxy organism, Desulfotalea psychrophila LSv54. FIG. 14B shows the Group 13 microcompartment also is present on other organisms.

FIG. 15A shows the microcompartment gene cluster found in Group 14 proxy organism, Desulfovibrio desulfuricans G20. FIG. 15B shows the Group 14 microcompartment also is present on other organisms.

FIG. 16A shows the microcompartment gene cluster found in Group 15 proxy organism, Alkaliphilus metalliredigens QYMF. FIG. 16B shows the Group 15 microcompartment also is present on other organisms.

FIG. 17A shows the microcompartment gene cluster found in Group 16 proxy organism, Alkaliphilus metalliredigens QYMF. FIG. 17B shows the Group 16 microcompartment also is present on other organisms.

FIG. 18 shows the microcompartment gene cluster found in Group 17 proxy organism, Leptotrichia buccallis.

FIG. 19A shows the microcompartment gene cluster found in Group 18 proxy organism, Salmonella typhimurium LT2. FIG. 19B shows the Group 18 microcompartment also is present on other organisms.

FIG. 20A shows the microcompartment gene cluster found in Group 19 proxy organism, Salmonella typhimurium LT2. FIG. 20B shows the Group 19 microcompartment also is present on other organisms.

FIG. 21 shows the microcompartment gene cluster found in Group 20 proxy organism, Clostridium kluveryi.

FIG. 22 shows the microcompartment gene cluster found in Group 21 proxy organism, Bacteroides capillosus.

FIG. 23 shows the microcompartment gene cluster found in Group 22 proxy organism, Opitutus terrae PB90-1.

FIG. 24 shows the microcompartment gene cluster found in Group 23 proxy organism, Chloroherpeton thalassium ATCC 35110.

FIG. 25 shows the microcompartment gene cluster found in Group 24A proxy organism, Thiomicrospira crunogena XCL-2.

FIG. 26 shows the microcompartment gene cluster found in Group 24B proxy organism, Prochlorococcus marinus MIT 9313.

### DETAILED DESCRIPTION OF THE PREFERRED EMBODIMENT

### Introduction

Carboxysome-like compartments (bacterial microcompartments) are currently found to be widespread in bacteria for various metabolic functions—many unknown.

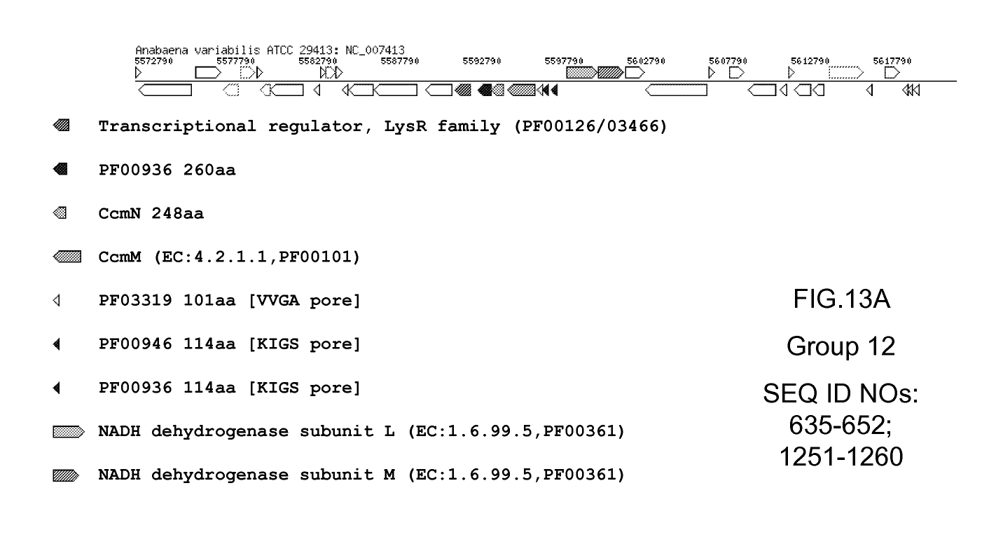

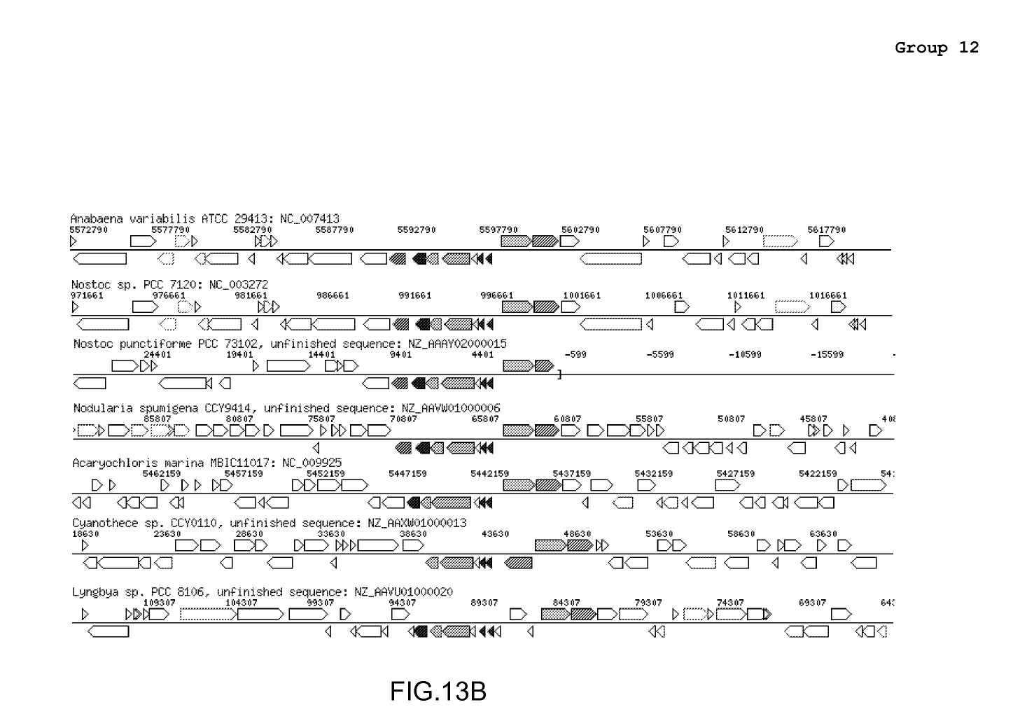

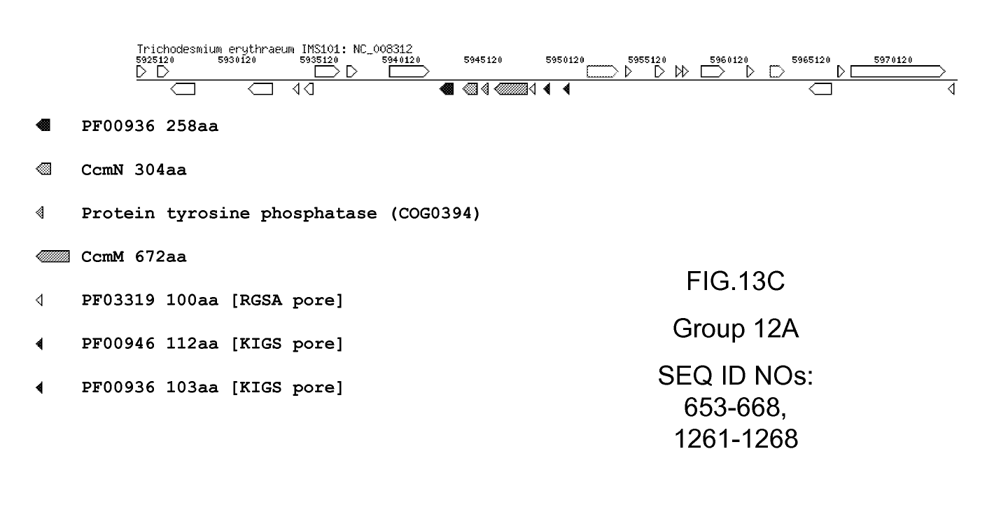

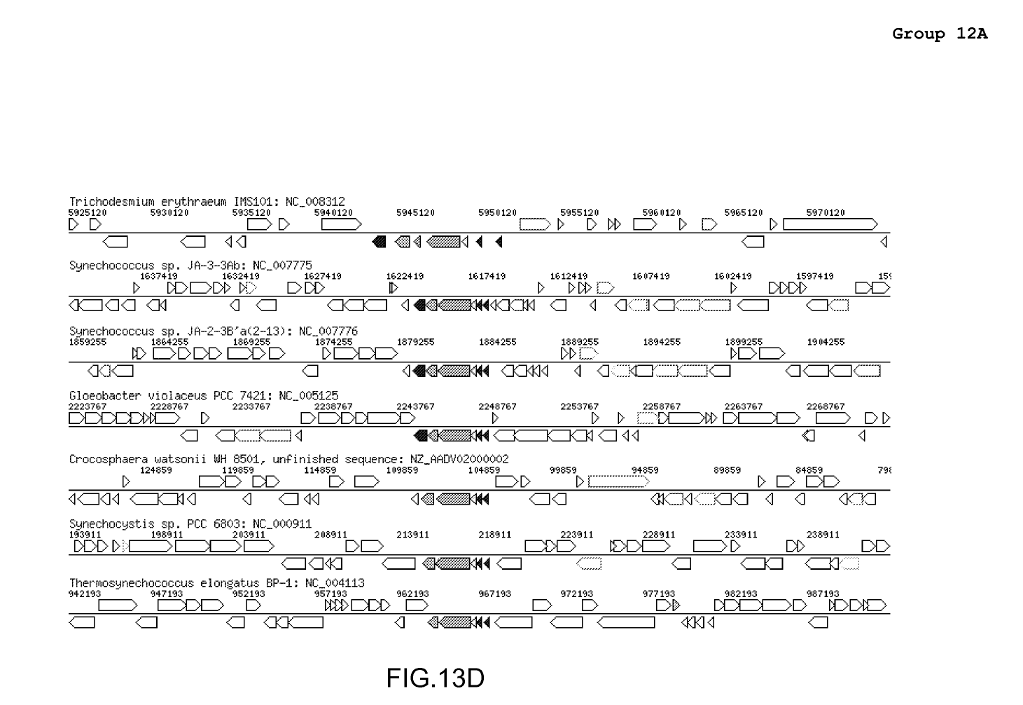

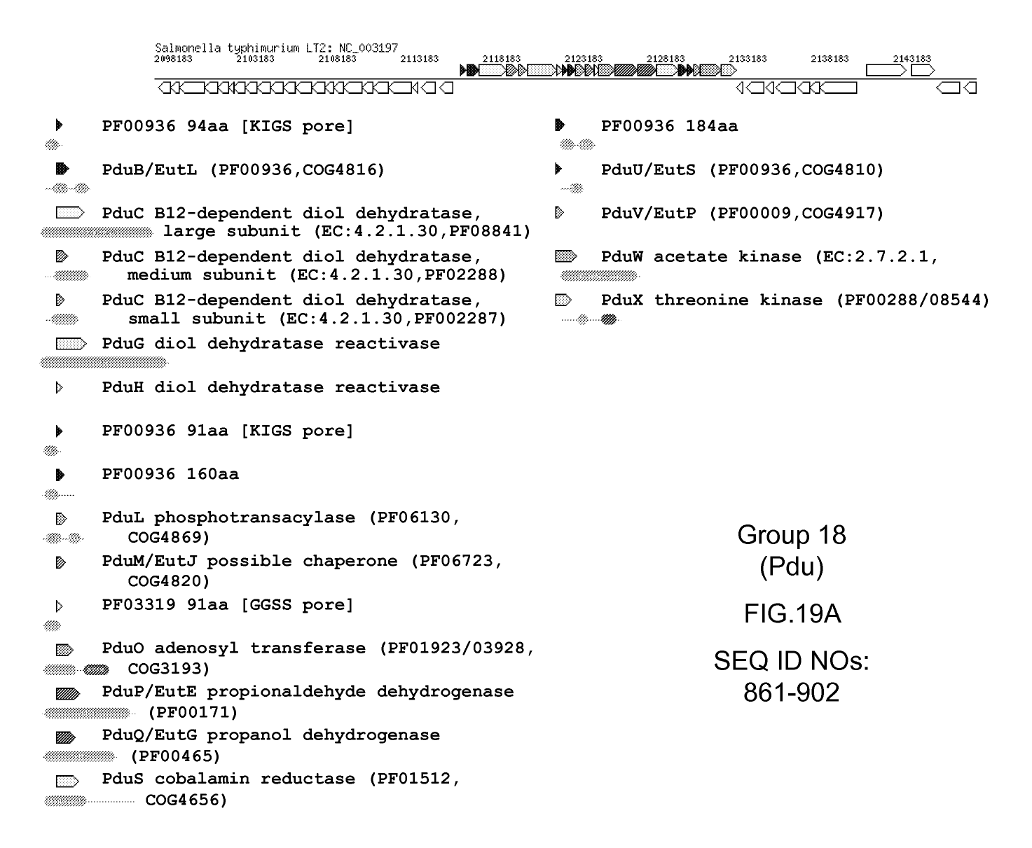

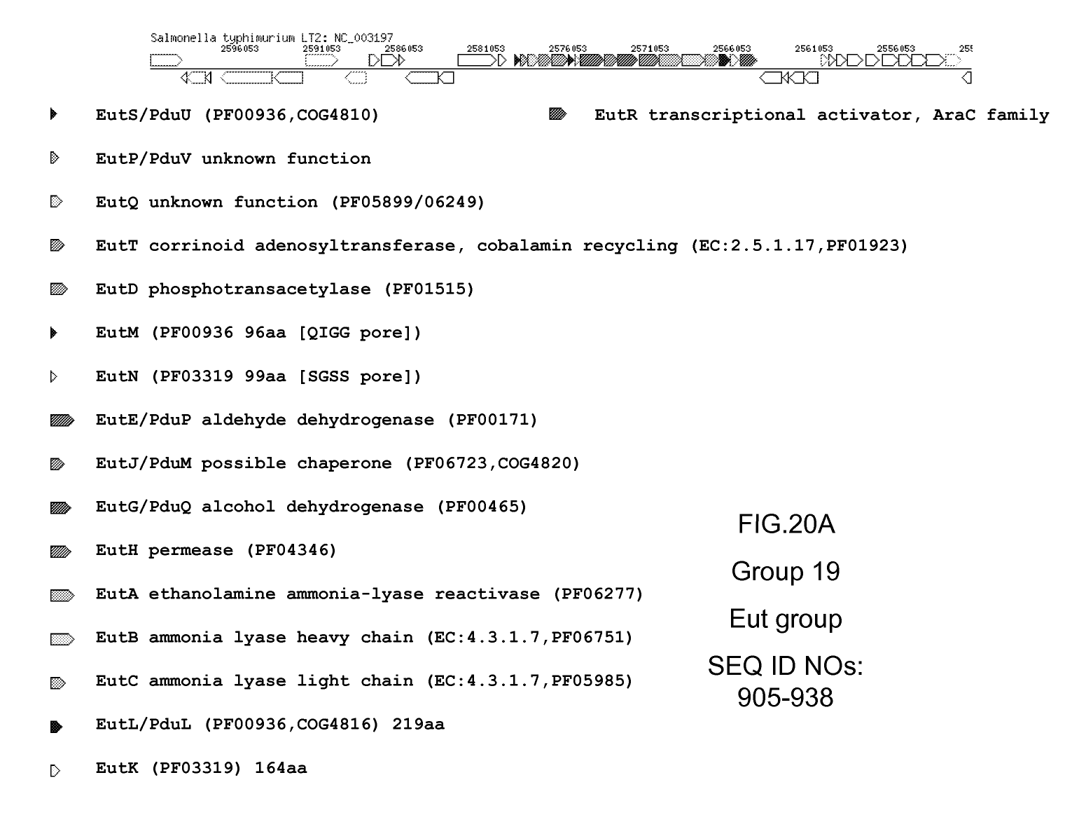

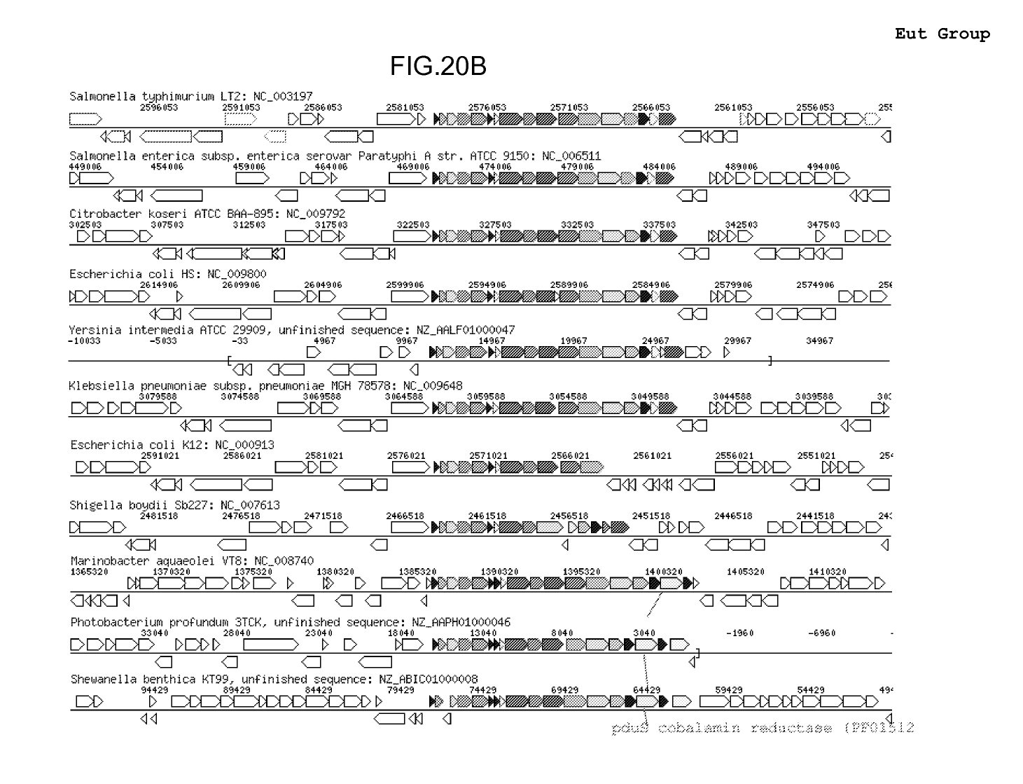

The prototypical bacterial microcompartment is the carboxysome, a bacterial polyhedral organelle which increases the efficiency of CO₂ fixation by encapsulating RuBisCO and carbonic anhydrase and other proteins. Carboxysomes can be divided into two types: alpha-type carboxysomes and beta-type carboxysomes (FIGS. 13, 25, 26). In addition to carboxysomes there are other experimentally characterized bacterial microcompartments that contain shell proteins homologous to those in the carboxysome; these include pdu bacterial microcompartments (FIG. 19A,B) involved in coenzyme B12-dependent degradation of 1,2-propanediol and eut bacterial microcompartments (FIG. 20A, B) involved in the cobalamin-dependent degradation of ethanolamine. Structural evidence shows that several carboxysome shell proteins and their homologs (e.g. Csos1A, D CcmK1,2,4, and PduU, EutL; collectively members of Pfam00936) exist as hexamers or pseudohexamers which might further assemble into extended, tightly packed layers hypothesized to represent the flat facets of the polyhedral organelles outer shell. It has been suggested that other homologous proteins in this family might also form hexamers and play similar functional roles in the construction of their corresponding organelle outer shell.

EutN_CcmL: Ethanolamine utilisation protein and carboxysome structural protein domain family (collectively, members of Pfam03319). Beside the Escherichia coli ethanolamine utilization protein EutN and the Synechocystis sp. carboxysome (beta-type) structural protein CcmL, this family also includes alpha-type carboxysome structural proteins CsoS4A and CsoS4B (previously known as OrfA and OrfB), propanediol utilization protein PduN, and some hypothetical homologous of various bacterial microcompartments. It is interesting that both carboxysome structural proteins CcmL and CsoS4A assemble as pentamers in the crystal structures, which might constitute the twelve pentameric vertices of a regular icosahedral carboxysome or otherwise introduce curvature into a micrompartment shell. However, the reported EutN structure is hexameric rather than pentameric. The absence of pentamers in Eut microcompartments might lead to less-regular icosahedral shell shapes. Due to the lack of structure evidence, the functional roles of the CsoS4A adjacent paralog, CsoS4B, and propanediol utilization protein PduN are not yet clear.

With these observations in mind and while cataloging/characterizing all bacterial microcompartment components, it was realized that these microcompartment components can be combined in novel ways or used as protein scaffolds to engineer new or enhanced active site capabilities thereby generating customized catalysis in a module,

For example, by encapsulating the enzymes necessary for this process within a protein shell, the propanediol utilization (pdu) microcompartment presumably protects the cell from propionaldehyde, a toxic intermediate. Likewise, microcompartments are formed in some enteric bacteria (including Salmonalla enterica and E. coli) when grown in the presence of ethanolamine. The ethanolamine utilization (eut) microcompartment is thought to sequester acetaldehyde, an intermediate in the degradation of ethanolamine, and might serve to either protect cells from the toxic effects of acetylaldehyde or to help retain this volatile intermediate, thereby preventing the loss of fixed carbon. The microcompartments that are formed during growth on 1,2-propanediol or ethanolamine seem to be less uniform in size and more irregular geometrically than carboxysome microcompartments, but it seems likely that they are constructed according to similar architectural principles, based on the homology between components of their shells. Two reviews written by one of the authors describes such interest in carboxysome compartments in Yeates, T. O., Kerfeld, C. A., Heinhorst, S., Cannon, G. C. and Shively, J. Protein-Based Organelles in Bacteria: Carboxysomes and Related Microcompartments. Nat. Rev Microbiol. 2008 September; 6(9):681-91. Review, online on Aug. 4, 2008, and Kerfeld, C. A., Heinhorst, S. and Cannon, G. C. Bacterial Microcompartments. Annual Review of Microbiology, in press both of which are hereby incorporated by reference.

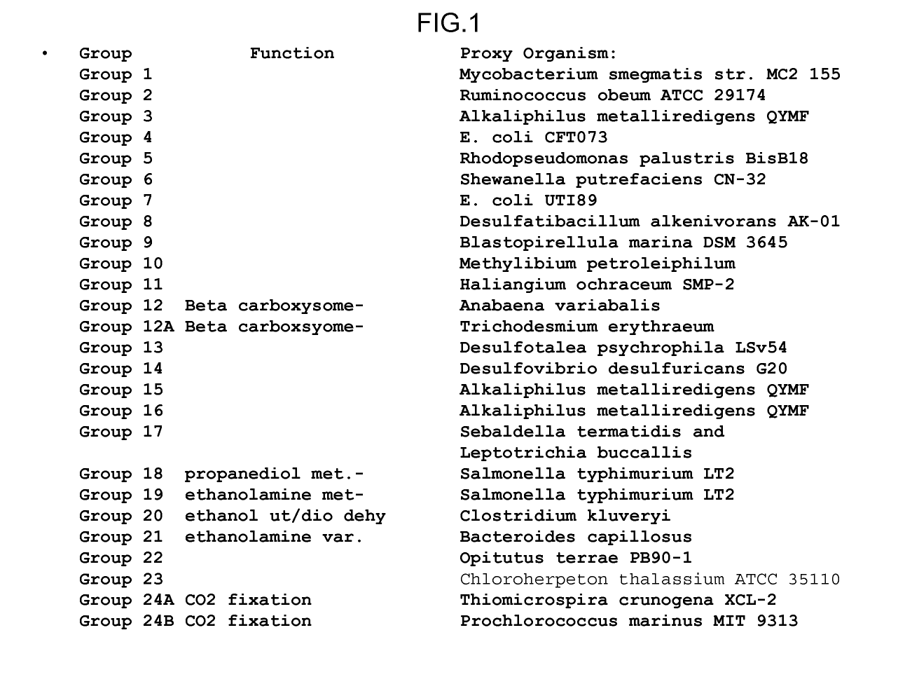

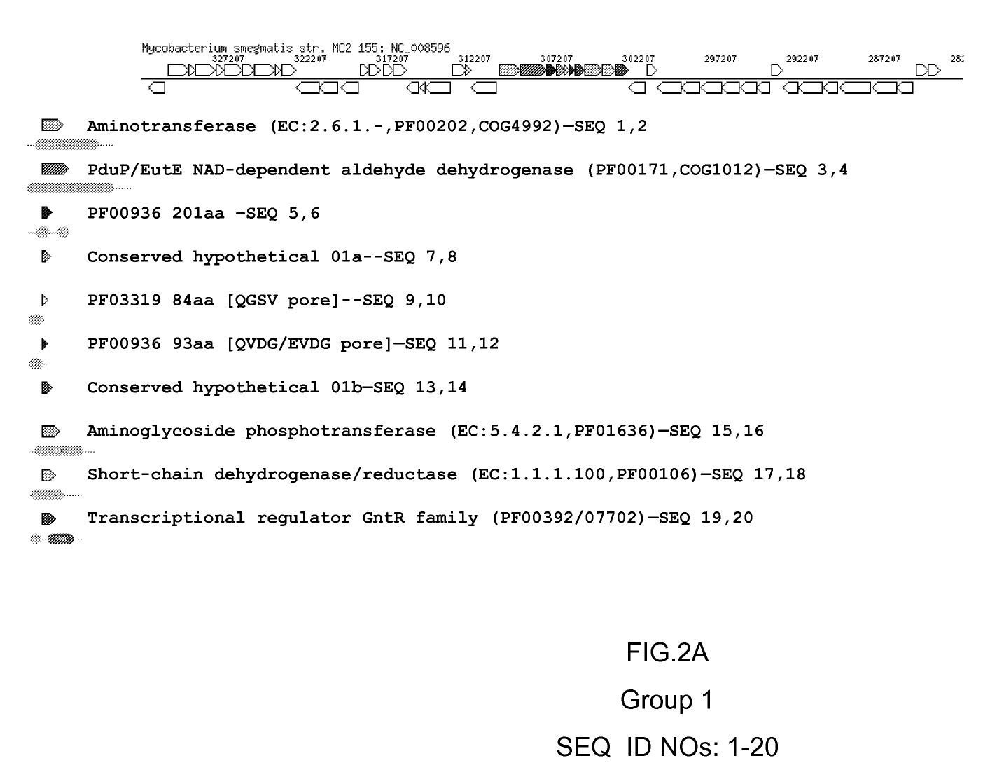

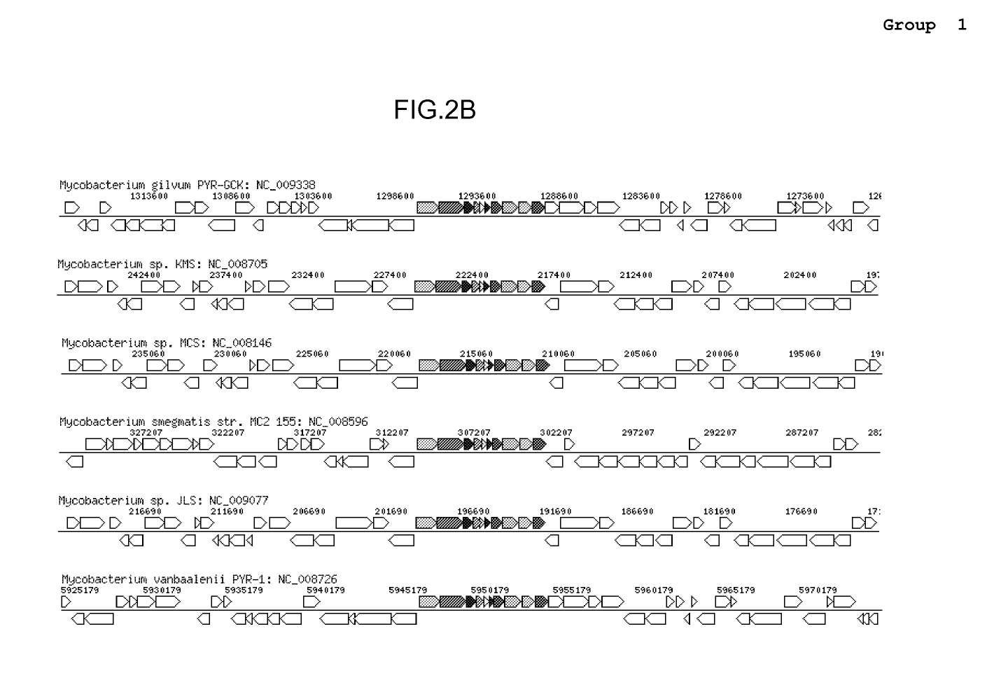

The pdu microcompartment and its numerous proteins and enzymes have been functionally characterized (FIG. 1D, FIG. 2B) by others in Bobik, T. A. Polyhedral organelles compartmenting bacterial metabolic processes. Appl. Microbiol. Biotechnol. 70, 517-525 (2006) and Havemann, G. D. & Bobik, T. A. Protein content of polyhedral organelles involved in coenzyme B12-dependent degradation of 1,2-propanediol in Salmonella enterica serovar Typhimurium LT2. J. Bacteriol. 185, 5086-5095 (2003). Purified and characterized the Pdu microcompartment, hereby incorporated by reference. Interestingly, the models for operation of the pdu microcompartment require movement of bulky molecules such as ATP and B₁₂ cofactors across the shell, raising further questions about molecular transport.

By taking naturally occurring components of bacterial microcompartments and modifying (e.g. altering active sites—essentially using the known encapsulated protein as a scaffold) and/or recombining them one can design new or enhanced bacterial microcompartments. These can be transferred among organisms (bacteria, plants, algae) using basic molecular techniques, followed by adaptive evolution to optimize phenotype. Alternatively, the modules are stable in solution or can be engineered to be (via reversible bonds/crosslinks) stable in solution, thus carrying out catalysis in cell free, non biological systems.

In another embodiment, one can engineer new metabolic modules (essentially organelles of specific function) into bacteria and thereby providing a new approach to designing and optimizing catalysis in solution. This is a way of bringing groups of enzymes that are functionally related into an organism or into solution. By delivering the enzymes encapsulated in the module, it is possible to introduce new functions that might otherwise be toxic to the cell, or incompatible with other aspects of cellular metabolism. Based on the design principles of naturally occurring metabolic modules, the naturally occurring assemblies of interior components and shell, we will be able to deliver groups of enzymes that are already (partially) optimized with respect to intermolecular interactions.

The present methods allow one to add new metabolic capabilities to bacteria, plants and algae, to carry out cell-free catalysis in solution that can be controlled by manipulating the microcompartment structure and organization (e.g. disassociating the catalytic microcompartment after catalytic reaction has reached a desired endpoint), and the enhancement of existing potentials of bacteria, plants and algae (e.g., increase RuBisCO activity in photosynthetic eukaryotes by adding microcompartment shell genes).

This could be used for any application in which bacteria play a role, including but not limited to, biomass conversion, bioreactors. One could use this to enhance the core metabolism of the bacterium (to make it grow better) or to introduce new functions (such as the production of 3-HPA or additional acetyl CoA) to an organism to increase its repertoire of functions

### DEFINITIONS

The term “bacterial microcompartment” as used herein is intended to describe and include genes with sequence or structural homology to the conserved bacterial microcompartment domains pfam00936 and/or pfam03319 along with any other genes that are associated or identifiable as in a gene cluster with these pfam00936 and/or pfam03319 homologs or are implicated microcompartment proteins by co-regulation with microcompartment genes and may encode proteins and/or enzymes having metabolizing activity. The term “gene cluster” or “cluster” or “cluster or genes” as used herein is intended to describe and include genes which are contiguous and generally not separated by more than about 300 bp from one another, but may include some genes which are distal in a genome but co-regulated or co-expressed with the genes found in the gene cluster. While many of the bacterial microcompartments are found in contiguous gene clusters, it is recognized that there may be multiple clusters within a genome, or alternatively, or in addition, many organisms that have gene clusters will also have scattered isolated genes that may also be co-regulated and can be incorporated into the bacterial microcompartment. The scattered genes may have been more recently acquired as it may be that once a bacteria acquires a BMC gene cluster, it can readily pick up and retain genes that could be co-expressed in the microcompartment although the gene may physically reside elsewhere in the genome.

In one embodiment, the cluster of genes containing one or more occurrences of Pfam00936 and/or Pfam03319 wherein all contiguous genes are not greater than about 300 bp from one another or are distal in the genome (including in plasmids), but co-regulated/expressed with bacterial microcompartment genes. Thus, in another embodiment, an expression cassette comprising a nucleic acid molecule comprising a cluster of bacterial compartment genes.

As used herein, the term, “host cell,” refers to any cell that can be transformed by foreign DNA where the foreign DNA may be a plasmid or vector containing a gene and the gene can be expressed in the cell. The host cell can be a cell from an organism, for example, microbial, including bacterial, fungal, and viral, plant, animal, or mammalian.

As used herein, the term, “library,” “clone library” or “genomic library” refers to a set of clones containing DNA fragments randomly generated by fragmentation of a genome or large DNA fragment, inserted into a suitable plasmid vector and cloned into a suitable host organism, such as E. coli. Sequencing of clones in a library involves carrying out sequence reactions to sequence the beginning and the end of the DNA fragment inserted into each sequenced clone, also referred to as “end sequences”, or “reads”. The genome or large DNA fragments may be from any eukaryote, including human, mammal, plant or fungus, or prokaryote, including bacteria, virus or archaea.

As used herein, the term “toxic” when used to define a gene, refers to a gene whose expression product inhibits the growth of microorganisms, such as bacteria and archaea. For example, a toxic gene can be a gene which when expressed in a host cell, causes the host cell to become nonviable or causes cell death, and is thus “toxic” to the cell.

As used herein, the term “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

As used herein, the terms “polypeptide” and “protein” and in some instances “enzyme(s)” are used interchangeably and are intended to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques. For example, a truncated protein of the invention can be produced by expression of a recombinant nucleic acid of the invention in an appropriate host cell, or alternatively by a combination of ex vivo procedures, such as protease digestion and purification, or in-vitro peptide synthesis. When referring to an enzyme, generally they are proteins having or exhibiting some metabolizing or catalytic activity.

As used herein, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. One of skill in the art will recognize that variants of the nucleic acids of the invention will be constructed such that the open reading frame is maintained. For polynucleotides, conservative variants include those sequences that, because of the degeneracy of the genetic code, encode the amino acid sequence of one of the microcompartment, shell proteins, proteins or enzyme polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode an microcompartment protein of the invention. Generally, variants of a particular polynucleotide of the invention will have at least about 30$, 35%, 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs. Where any given pair of polynucleotides of the invention is evaluated by comparison of the percent sequence identity shared by the two polypeptides they encode, the percent sequence identity between the two encoded polypeptides is at least about 30%, 35%, 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, that is, microcompartment activity as described herein. Such variants may result from, for example, genetic polymorphism or from human manipulation. Biologically active variants of a native microcompartment protein of the invention will have at least about 30%, 35%, 40%, 45%, 50%, 55%, 60%, 65%, 70%, 75%, 80%, 85%, more preferably 90%, 91%, 92%, 93%, 94%, 95%, 96%, 97%, 98%, 99% or more sequence identity to the amino acid sequence for the native protein as determined by sequence alignment programs. A biologically active variant of a protein of the invention may differ from that protein by as few as 1-15 amino acid residues, as few as 1-10, such as 6-10, as few as 5, as few as 4, 3, 2, or even 1 amino acid residue.

As used herein, a gene is said to have homology if there is at least about 30%, 35%, 40%, 45%, 50%, 55%, 60%, 65%, 70%, 75%, 80%, 85%, more preferably 90%, 91%, 92%, 93%, 94%, 95%, 96%, 97%, 98%, 99% or more sequence identity to the amino acid sequence for the native protein as determined by sequence alignment programs (such as BLAST) or if there is structural similarity as determined by three-dimensional structural superposition algorithms such as SUPERPOSE or superposition applications in PYMOL.

The proteins of the invention may be altered in various ways including amino acid substitutions, deletions, truncations, and insertions. Methods for such manipulations are generally known in the art. For example, amino acid sequence variants and fragments of the microcompartment proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences and their variants as well as mutant forms. Likewise, the proteins of the invention encompass naturally occurring proteins as well as variations and modified forms thereof. Such variants will continue to possess the desired microcompartment activity.

In nature, some polypeptides are produced as complex precursors which, in addition to targeting labels such as the signal peptides for example in chloroplasts, also contain other fragments of peptides which are removed (processed) at some point during protein maturation, resulting in a mature form of the polypeptide that is different from the primary translation product (aside from the removal of the signal peptide). “Mature protein” refers to a post-translationally processed polypeptide; i.e., one from which any pre- or propeptides present in the primary translation product have been removed. “Precursor protein” or “prepropeptide” or “preproprotein” all refer to the primary product of translation of mRNA; i.e., with pre- and propeptides still present. Pre- and propeptides may include, but are not limited to, intracellular or extracellular localization signals. “Pre” in this nomenclature generally refers to the signal peptide. The form of the translation product with only the signal peptide removed but no further processing yet is called a “propeptide” or “proprotein.” The fragments or segments to be removed may themselves also be referred to as “propeptides.” A proprotein or propeptide thus has had the signal peptide removed, but contains propeptides (here referring to propeptide segments) and the portions that will make up the mature protein. The skilled artisan is able to determine, depending on the species in which the proteins are being expressed and the desired intracellular location, if higher expression levels or higher microcompartment activity might be obtained by using a gene construct encoding just the mature form of the protein, the mature form with a signal peptide, or the proprotein (i.e., a form including propeptides) with a signal peptide. For optimal expression in plants or fungi, the pre- and propeptide sequences may be needed. The propeptide segments may play a role in aiding correct peptide folding.

As used herein in the specification and in the claims section that follows, the phrase “photosynthetic organism” includes organisms, both unicellular or multicellular, both prokaryotes or eukaryotes, both soil grown or aquatic, capable of producing complex organic materials, especially carbohydrates, from carbon dioxide using light as the source of energy and with the aid of chlorophyll and optionally associated pigment.

The method according to the present invention is effected by transforming cells of an organism with an expressible polynucleotide encoding a polypeptide encoding a bacterial microcompartment and in some embodiments, having a bicarbonate (HCO₃″) transporter activity.

As used herein in the specification and in the claims section that to follows, the term “transform” and its conjugations such as transformation, transforming and transformed, all relate to the process of introducing heterologous nucleic acid sequences into a cell or an organism. The term thus reads on, for example, “genetically modified”, “transgenic” and “transfected” or “viral infected” and their conjugations, which may be used herein to further described the present invention. The term relates both to introduction of a heterologous nucleic acid sequence into the genome of an organism and/or into the genome of a nucleic acid containing organelle thereof, such as into a genome of chloroplast or a mitochondrion.

As used herein in the specification and in the claims section that follows, the phrase “expressible polynucleotide” refers to a nucleic acid sequence including a promoter sequence and a downstream polypeptide encoding sequence, the promoter sequence is so positioned and constructed so as to direct transcription of the downstream polypeptide encoding sequence.

As used herein in the specification and in the claims section that follows, the term “polypeptide” refers also to a protein, in particular a transmembrane protein, which may include a transit peptide, and further to a post translationally modified protein, such as, but not limited to, a phosphorylated protein, glycosylated protein, ubiquitinylated protein, acetylated protein, methylated protein, etc.

As used herein in the specification and in the claims section that follows, the phrase “bicarbonate transporter activity” refers to the direct activity of a membrane integrated protein in transporting bicarbonate across a membrane in which it is integrated. Such a membrane can be the cell membrane and/or a membrane of an organelle, such as the chloroplast's outer and inner membrane. Such activity can be effected by direct expenditure of energy, i.e., ATP hydrolysis, which is available both in the cytoplasm and the chloroplast's stroma, or by co- or anti-transport, as effected by co- or antiporters while dissipating a concentration gradient of an ion across a membrane.

According to another aspect of the present invention there is provided a nucleic acid molecule for enhancing inorganic carbon fixation by a photosynthetic organism. The nucleic acid molecule according to this aspect of the present invention includes a polynucleotide encoding a polypeptide having a bicarbonate transporter activity.

As used herein in the specification and in the claims section that follows, the term “nucleic acid molecule” includes polynucleotides, constructs and vectors. The terms “construct” and “vector” may be used herein interchangeably.

### Selecting Bacterial Microcompartment Sequences and Groups

In one embodiment, a bacterial microcompartment catalog comprising a total of 1268 gene sequences encoding bacterial microcompartments, the proteins of each can be inserted into a host organism and if needed, expressed using an inducible expression system. The Sequence Listing attached and herein incorporated by reference shows the gene number, internal reference number and the corresponding sequence identifier for the nucleotide and protein sequences, along with the either GenBank Accession Number of each gene, or the GenBank Conserved Domain Number as noted in Table 3, wherein the contents and identities of the GenBank entry are incorporated by reference at the time of filing.

In another embodiment, a bacterial microcompartment catalog is provided in the Sequence Listing and the Figures. The entire catalog comprising 634 gene sequences encoding bacterial microcompartments, the proteins of each can be inserted into a host organism and if needed, expressed using an inducible expression system.

FIG. 1 and Table 1 shows the index of the catalog which is comprised of 32 main groups and subgroups of microcompartment clusters and organized by microcompartment function and proxy organism. Examples of proxy organisms include Mycobacterium smegmatis str. MC2 155, Ruminococcus obeum ATCC 29174, Alkaliphilus metalliredigens QYMF, E. coli CFT073, Rhodopseudomonas palustris B is B18, Shewanella putrefaciens CN-32, E. coli UTI89, Desulfatibacillum alkenivorans AK-01, Blastopirellula marina DSM 3645, Methylibium petroleiphilum, Haliangium ochraceum SMP-2, Anabaena variabalis, Trichodesmium erythraeum, Desulfotalea psychrophila LSv54, Desulfovibrio desulfuricans G20, Alkaliphilus metalliredigens QYMF, Sebaldella termatidis and Leptotrichia buccallis, Salmonella typhimurium LT2.

TABLE 1
Index of BMC catalog

| | SEQ ID | | |
|---|---|---|---|
| Group | NOS: | Function | Proxy Organism: |
| Group 1 | 1-20 | | Mycobacterium smegmatis |
| | | | str. MC2 155 |
| Group 1A | 21-44 | | Verminephrobacter eiseniae |
| | | | EF01-2 |
| Group 1B | 45-68 | | Rhodococcus sp. RHA1 |
| | | | plasmid pRHL2 |
| Group 2 | 69-98 | | Ruminococcus obeum |
| | | | ATCC 29174 |
| Group 3 | 99-146 | | Alkaliphilus metalliredigens |
| | | | QYMF |
| Group 3A | 147-176 | | Carboxydothermus |
| | | | hydrogenoformans Z-2901 |
| Group 4 | 177-234 | | E. coli CFT073 |
| Group 5 | 235-270 | | Rhodopseudomonas palustris |
| | | | BisB18 |
| Group 5A | 271-296 | | Clostridium novyi NT |
| Group 6 | 297-342 | | Shewanella putrefaciens |
| | | | CN-32 |
| Group 7 | 343-386 | | E. coli UTI89 |
| Group 8 | 387-436 | | Desulfatibacillum |
| | | | alkenivorans AK-01 |
| Group 8A | 437-482 | | Clostridium kluyveri |
| | | | DSM 555 |
| Group 8B | 483-534 | | Dethiosulfovibrio |
| | | | peptidovorans SEBR 4207, |
| | | | DSM 11002 |
| Group 9 | 535-560 | | Blastopirellula marina |
| | | | DSM 3645 |
| Group 10 | 561-608 | | Methylibium petroleiphilum |
| Group 11 | 609-634 | | Haliangium ochraceum |
| | | | SMP-2 |
| Group 12 | 635-652, | Beta | Anabaena variabalis |
| | 1251-1260 | carboxysome- | |
| Group 12A | 653-668, | Beta | Trichodesmium erythraeum |
| | 1261-1268 | carboxsyome- | |
| Group 13 | 669-714 | | Desulfotalea psychrophila |
| | | | LSv54 |
| Group 14 | 715-772 | | Desulfovibrio desulfuricans |
| | | | G20 |
| Group 15 | 773-814 | | Alkaliphilus metalliredigens |
| | | | QYMF |
| Group 16 | 815-860 | | Alkaliphilus metalliredigens |
| | | | QYMF |
| Group 17 | | | Sebaldella termatidis and |
| | 1055-1098 | | Leptotrichia buccallis |
| Group 18 | 861-902 | propanediol | Salmonella typhimurium |
| | | met.- | LT2 |
| Group 19 | 903-936- | ethanolamine | Salmonella typhimurium |
| | | met- | LT2 |
| Group 20 | 995-1054 | ethanol ut/ | Clostridium kluveryi |
| | | diodehy | |
| Group 21 | 1099-1196 | ethanolamine | Bacteroides capillosus |
| | | var. | |
| Group 22 | 1197-1232 | | Opitutus terrae PB90-1 |
| Group 23 | 1233-1250 | | Chloroherpeton thalassium |
| | | | ATCC 35110 |
| Group 24A | 937-970 | CO2 fixation | Thiomicrospira crunogena |
| | | | XCL-2 |
| Group 24B | 971-994 | CO2 fixation | Prochlorococcus marinus |
| | | | MIT 9313 |

FIGS. 2A, 3A, 4A, etc to 26A and also 13C show the legend and assign a color and shape for each enzyme or protein that comprises or has activity within a compartment in the Group proxy organism. FIGS. 2B, 3B, 4B, etc. to 20B and also 13D show the Group microcompartment cluster as observed in various other organisms.

For example, as seen in FIG. 13C, the Group 12A cluster of genes encodes a beta-carboxysome and comprised of the following genes: PF00936 258aa, CcmN 304aa, Protein tyrosine phosphatase (COG0394), CcmM 672aa, PF03319 100aa [RGSA pore], PF00936 112aa [KIGS pore], and PF00936 103aa [KIGS pore]. In another embodiment, elsewhere on the chromosome, further comprising genes encoding the large (Pfam00016/02788) and small (Pfam00101) subunits of RuBisCO, the RuBisCO chaperone, RbcX (Pfam02341) and additional shell (Pfam00936) proteins, which are components of assembly and structure of the carboxysome. The proxy organism is Trichodesmium erythraeum, but this compartment is also found in various other organisms as shown in FIG. 13D, in various forms.

Table 2 extends the information shown in Table 1 and shows the Group, Figure Number(s), SEQ ID Numbers, Representative organism, Potentially encapsulated reactions, Organism phenotypes, Enzymes (proposed from annotation), Proposed Reason for Encapsulation, and Additional Notes for a majority of the Groups shown in Table 1. Some of the Groups are combined where it may be that there is similar function or metabolizing activity provided by the microcompartment cluster of some Groups.

Thus, as shown in the Examples, in one embodiment, a custom metabolic microcompartment can be designed using the Groups and clusters of genes in the catalog presented herein to transform an organism or plant. Depending on what the level and type of activity and output is required in a transformed organism, one can provide the microcompartment shell proteins and interchangeably insert into the cluster any number of other enzymes and proteins from the catalog, to produce an expression cassette, which can then be used to transform an organism and thereby providing or enhancing custom metabolic activity.

In another embodiment, the expression cassette comprising the set of sequences comprising one of the Groups of genes as listed in Table 1. In one embodiment, the Groups of sequences are the following groups of sequences: SEQ ID NOS: 1-20, 21-44, 45-68, 69-98, 99-146, 147-176, 177-234, 235-270, 271-296, 297-342, 343-386, 387-436, 437-482, 483-534, 535-560, 561-608, 609-634, 635-652 and 1251-1260, 653-668 and 1261-1268, 669-714, 715-772, 773-814, 815-860, 1055-1098, 861-902, 903-936-, 937-970, 971-994, 995-1054, 1099-1196, 1197-1232, or 1233-1250.

Each of the 32 Groups of genes as listed in Table 1 (including the subgroups) is comprised of a cluster of genes, and the order of the genes in that cluster are found in other organisms. The Groups and the order and the sequences of the genes found in the cluster for each Group is as follows in Table 3. The functions are computationally-derived annotations. The direction of transcription is indicated in the corresponding Figure:

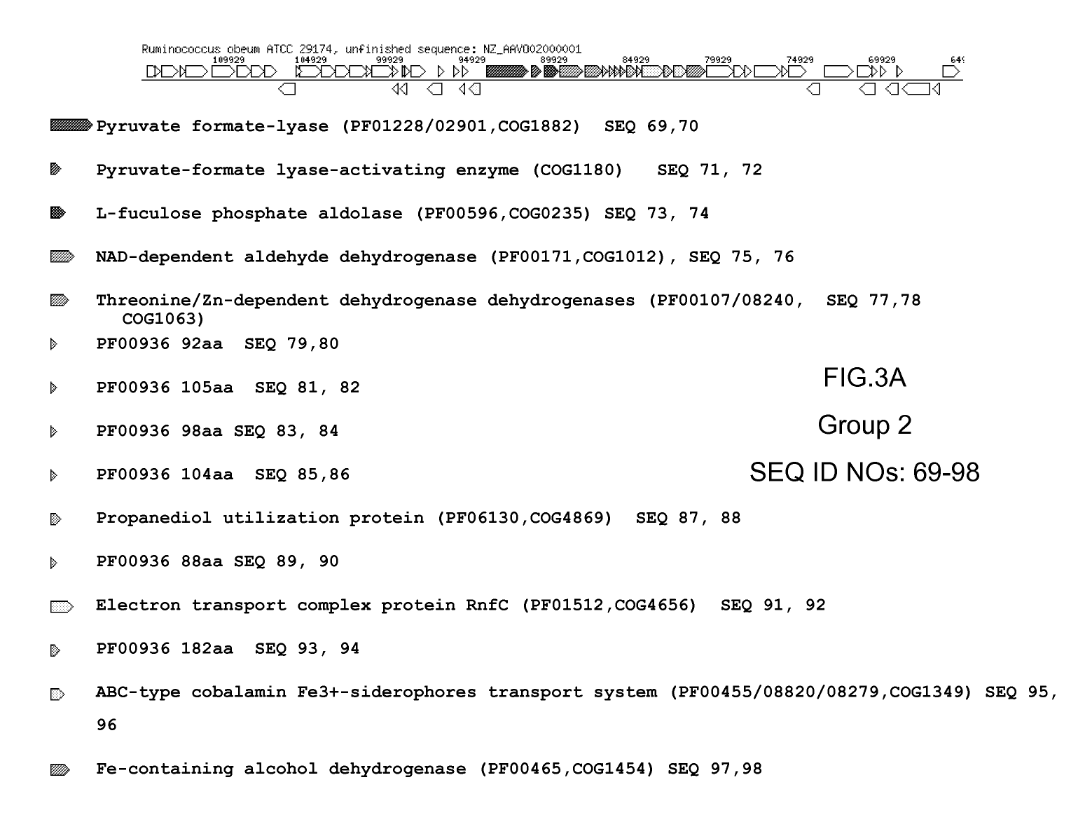

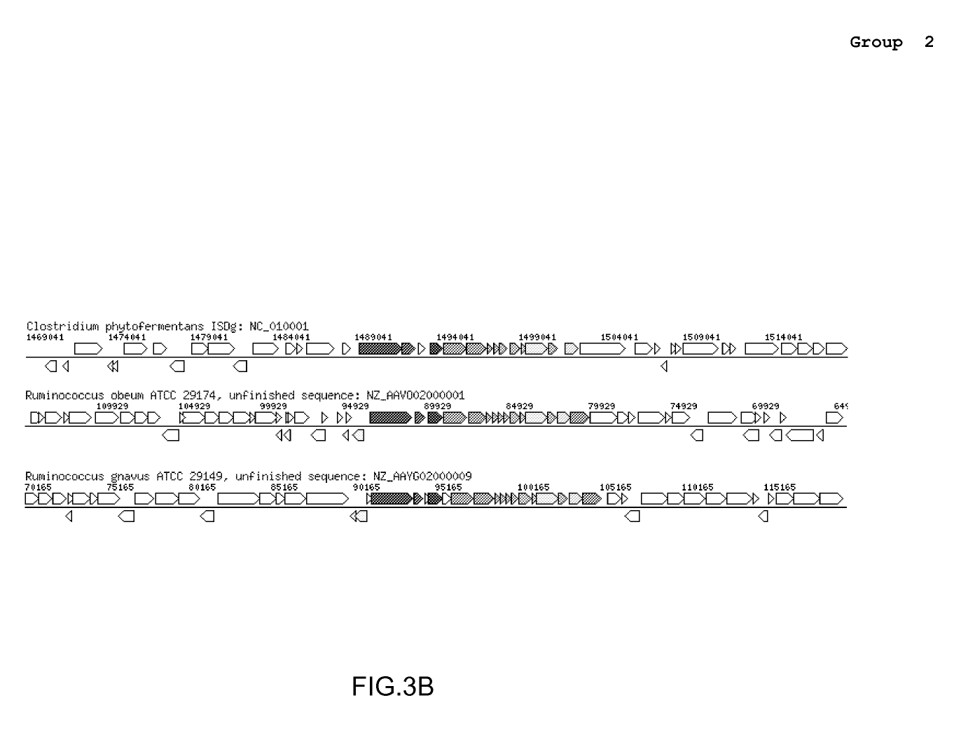

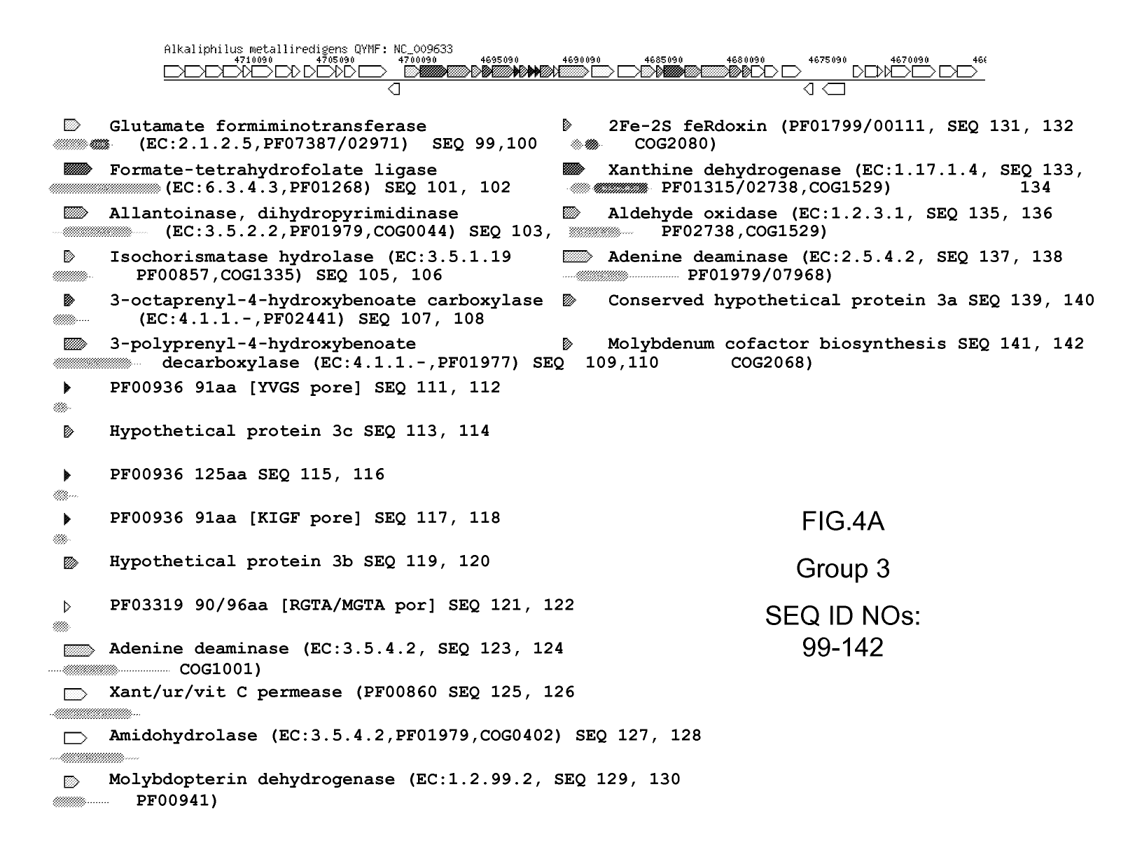

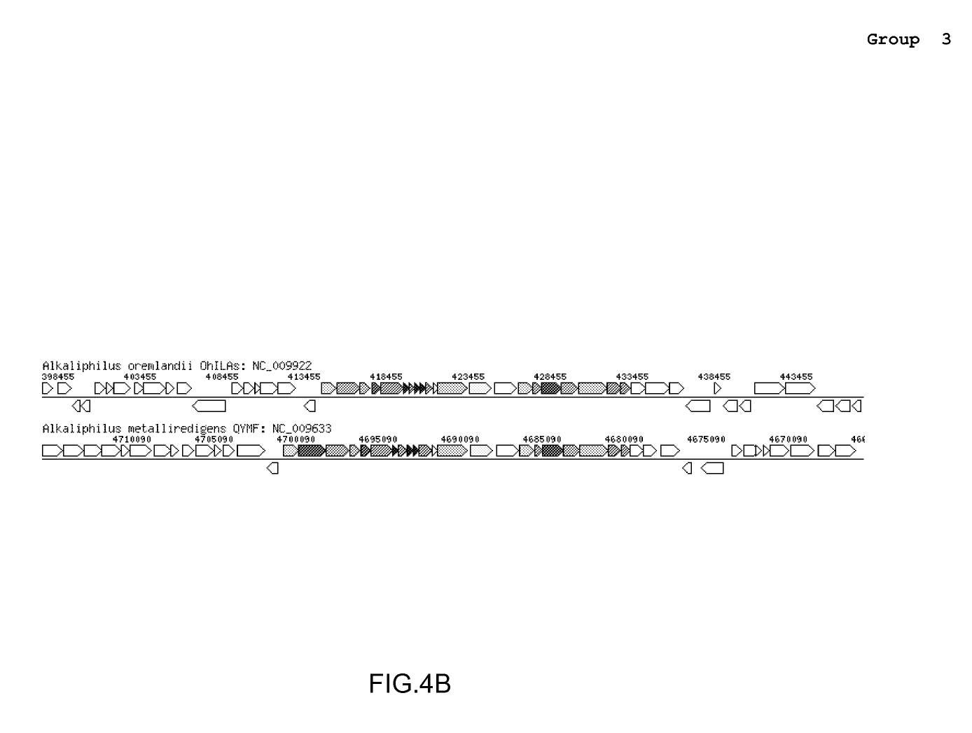

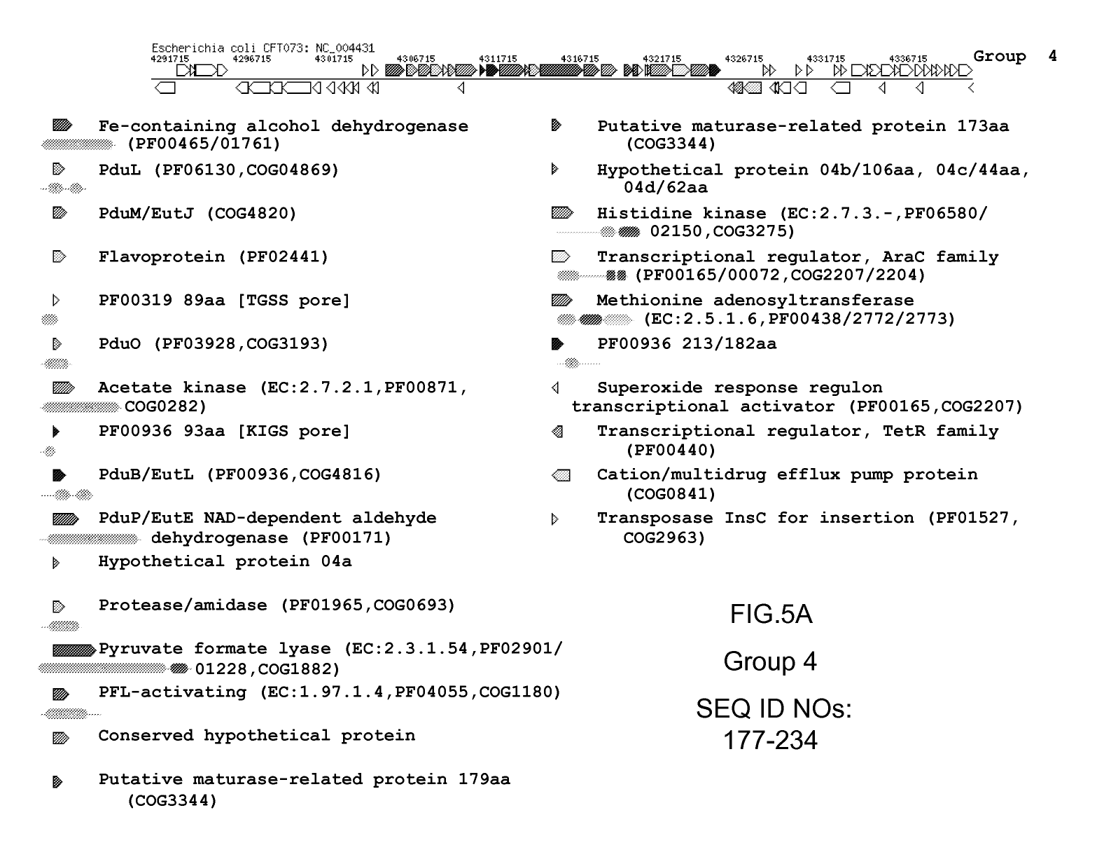

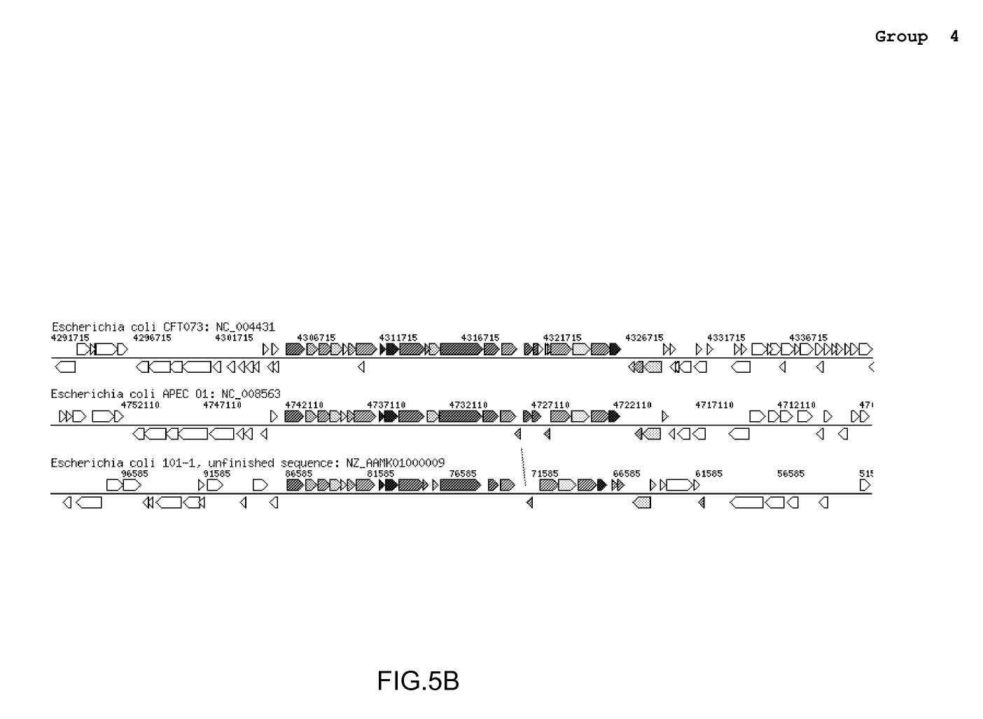

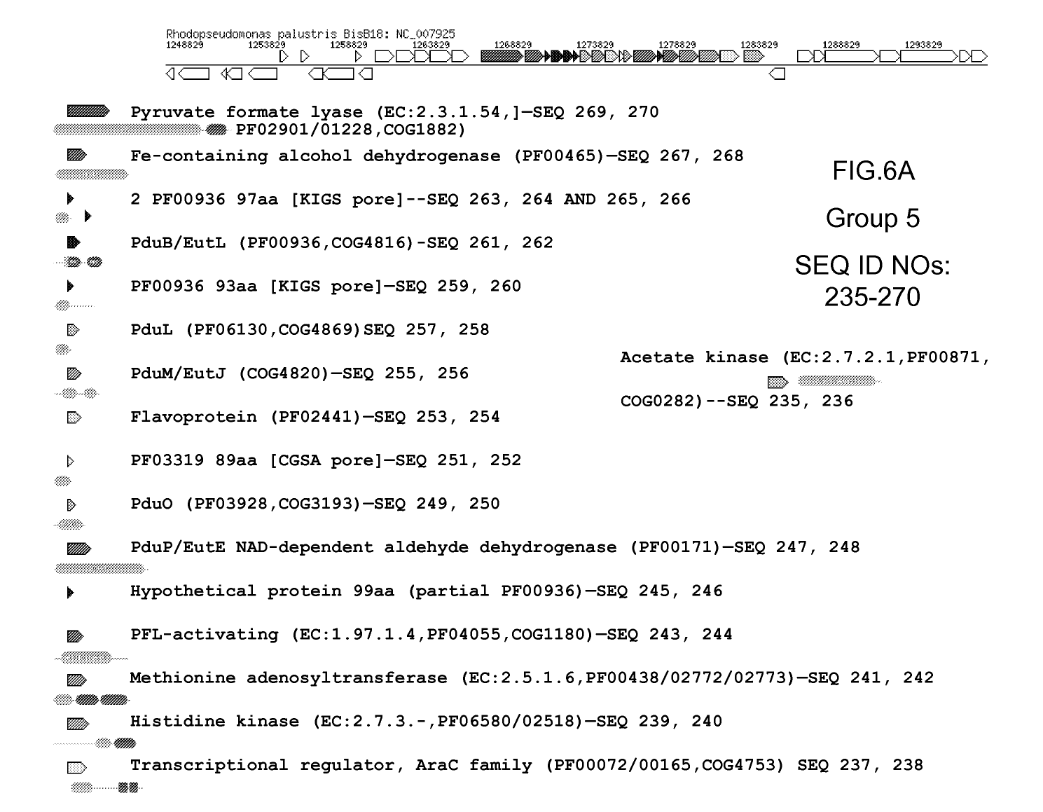

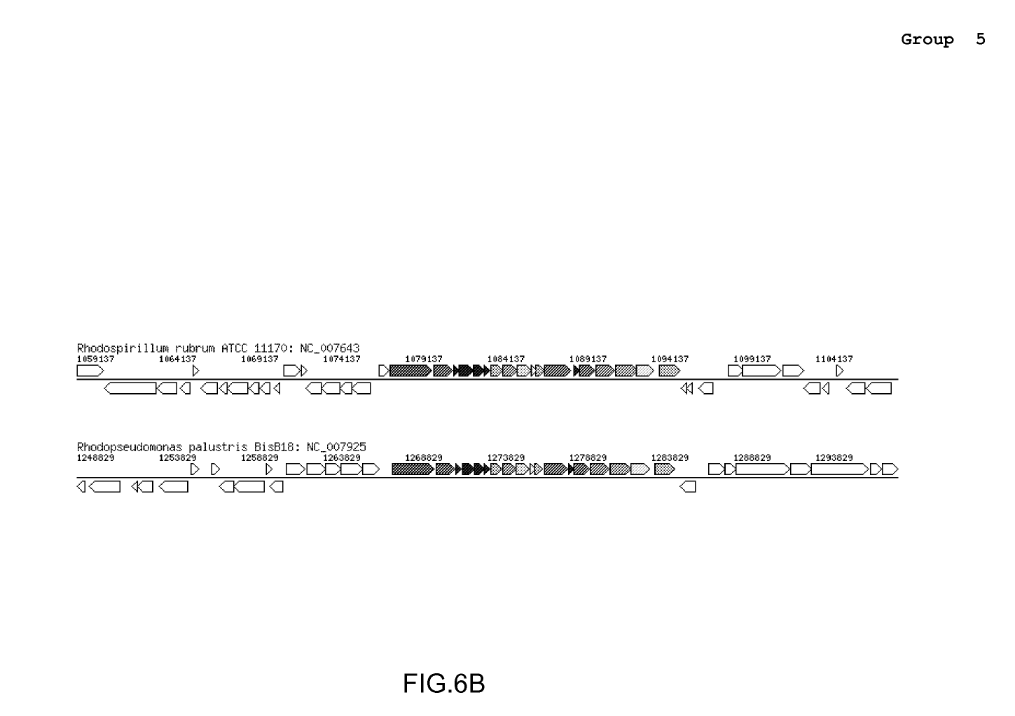

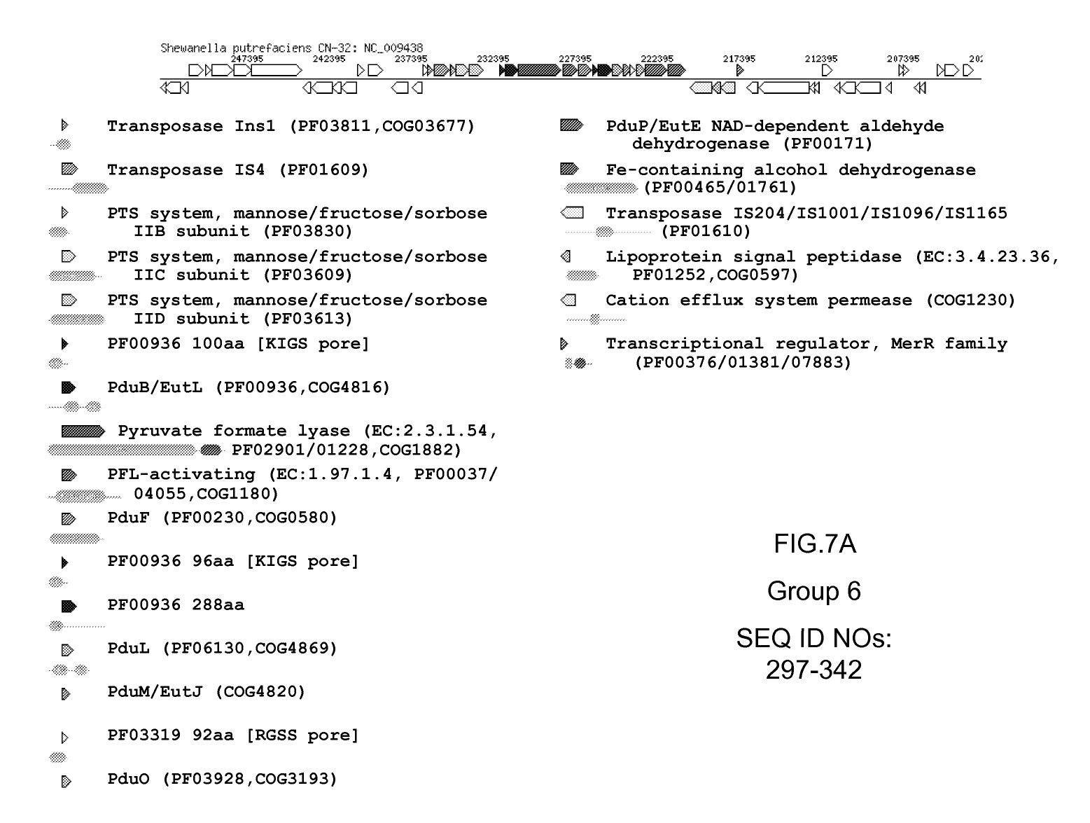

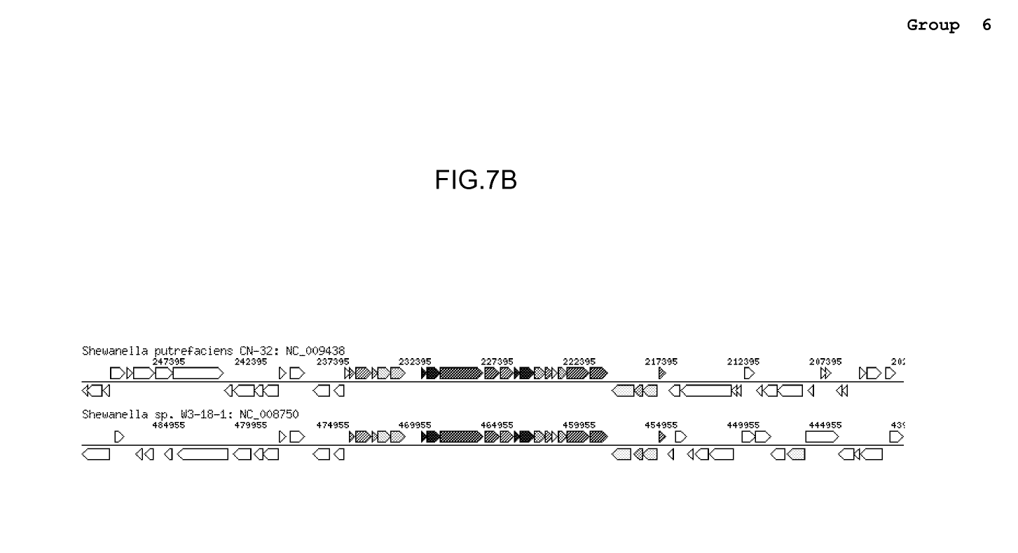

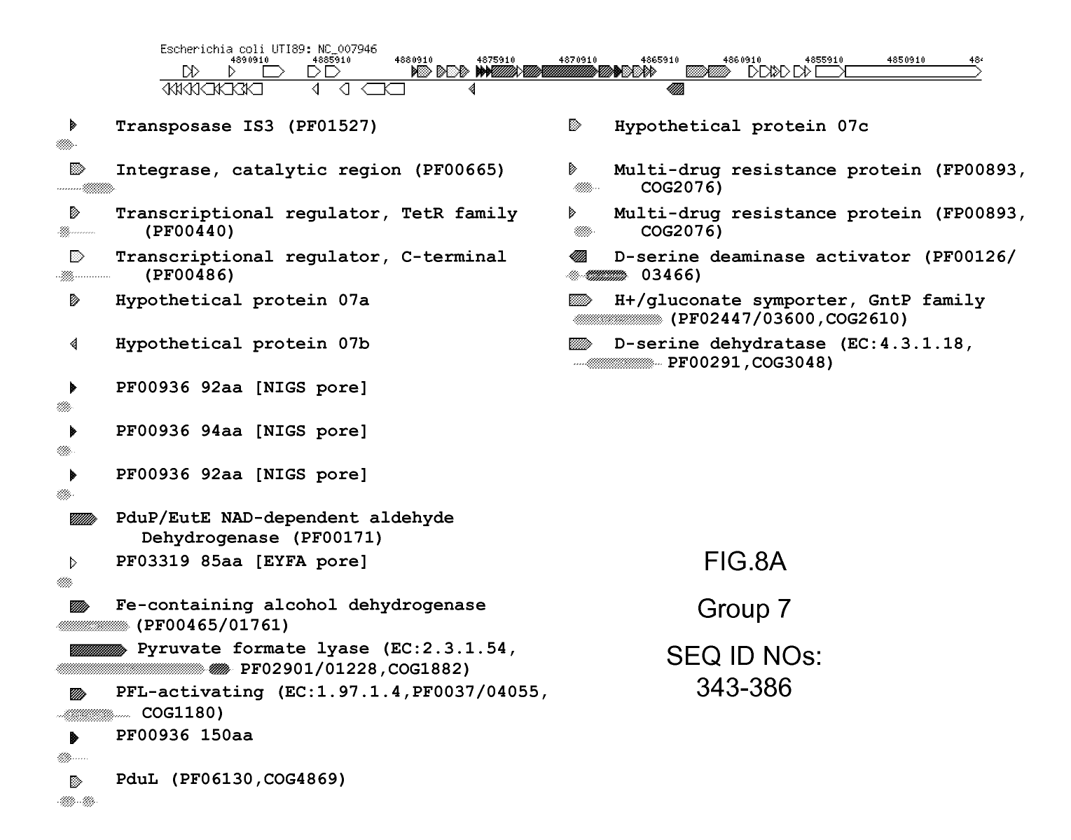

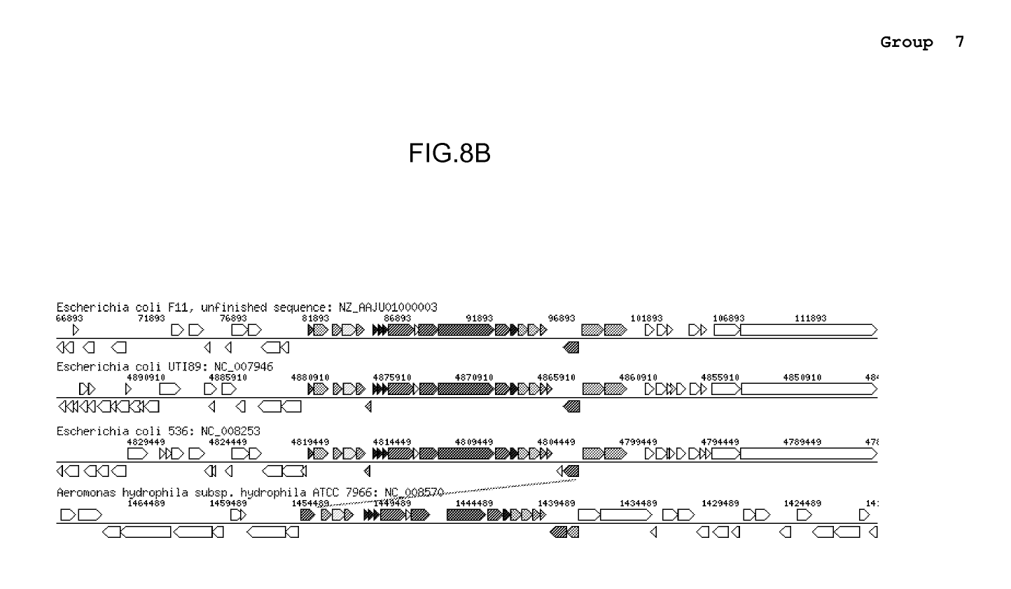

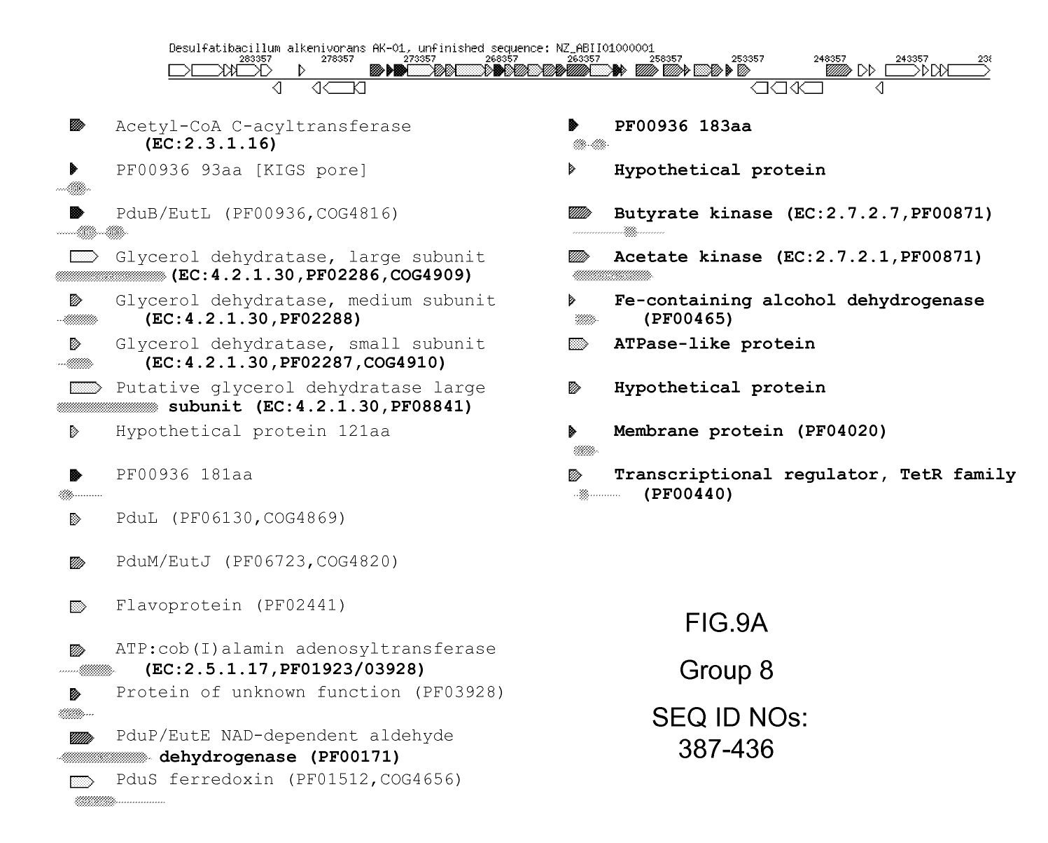

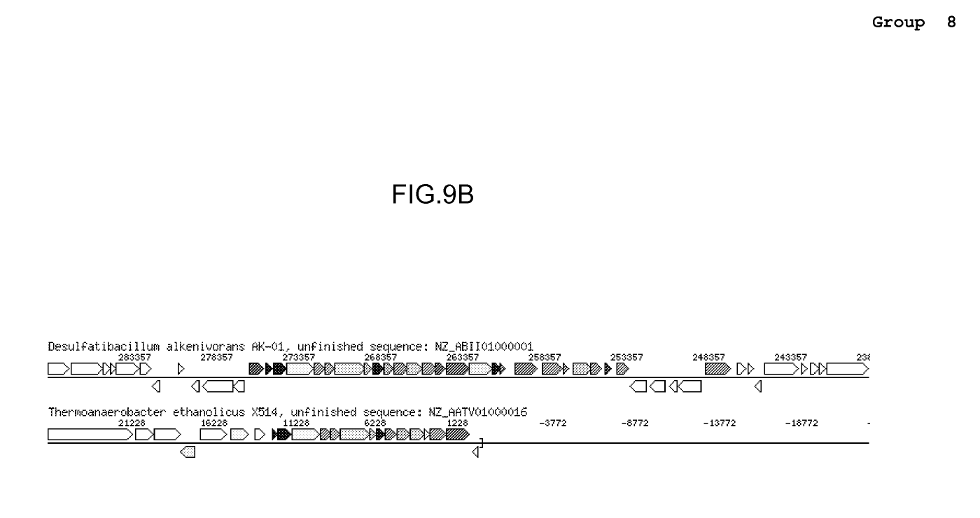

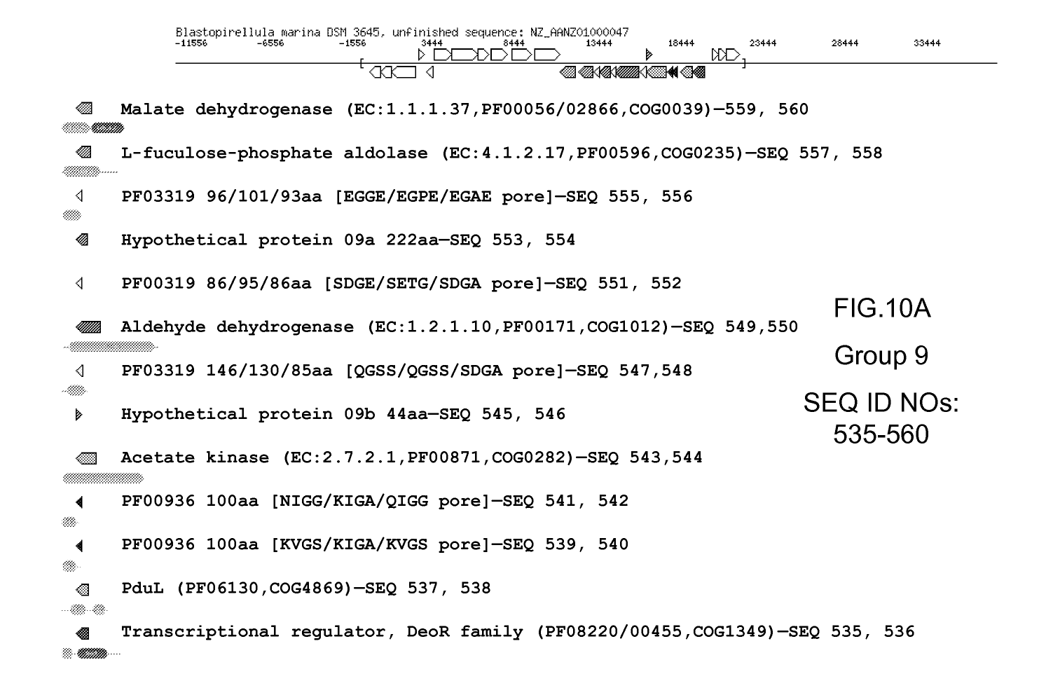

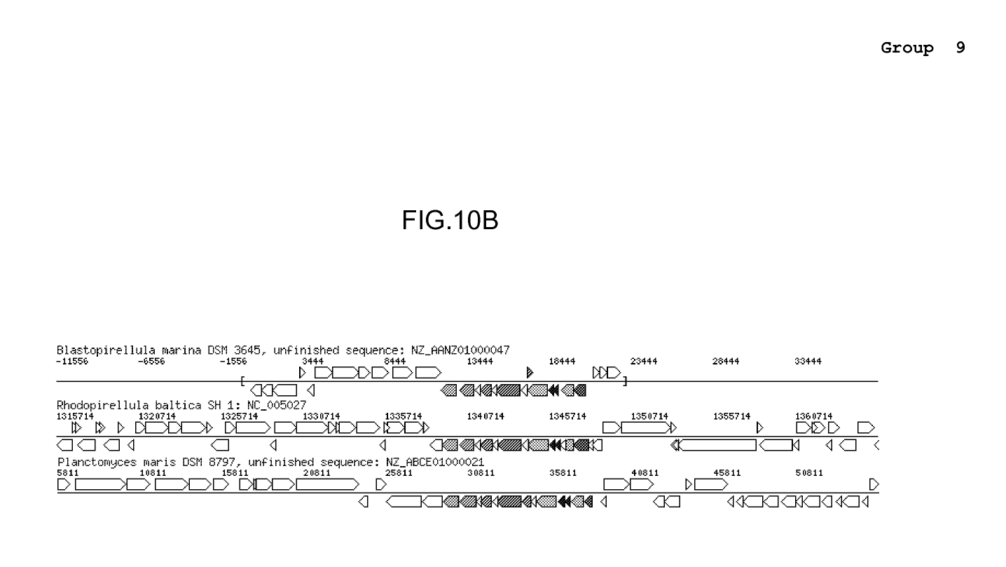

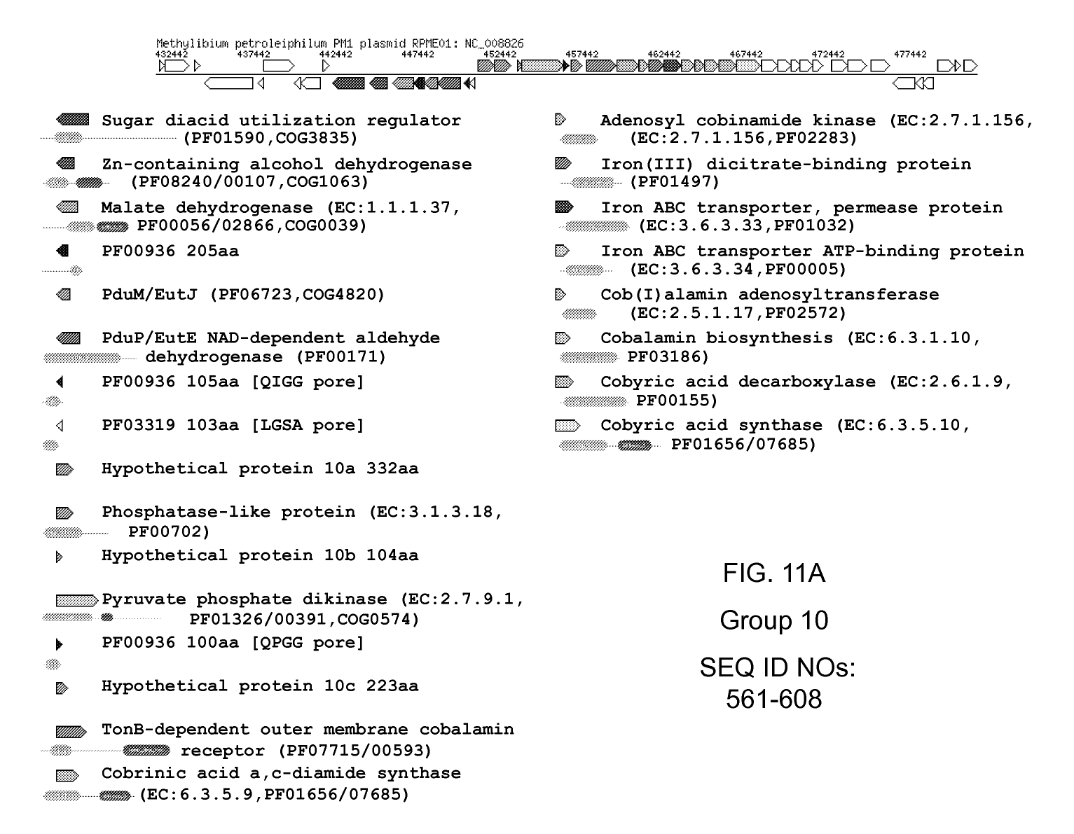

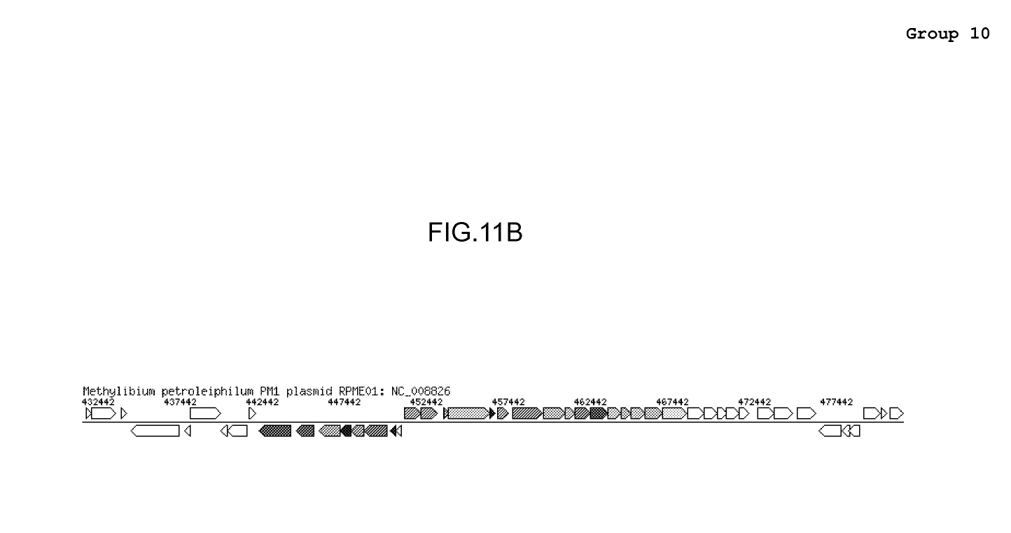

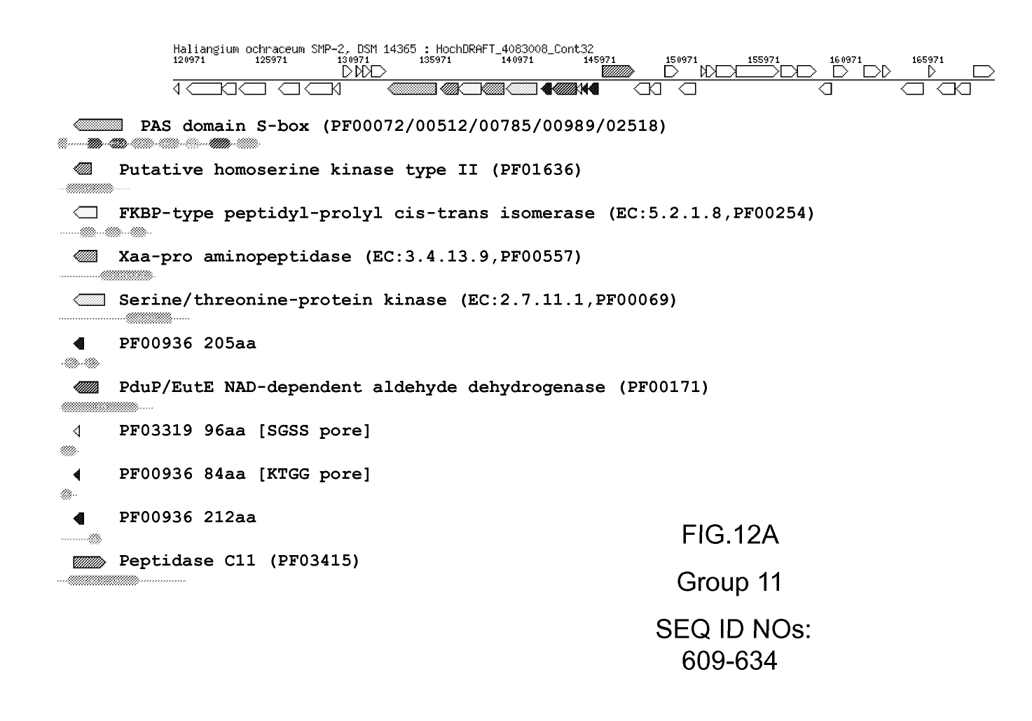

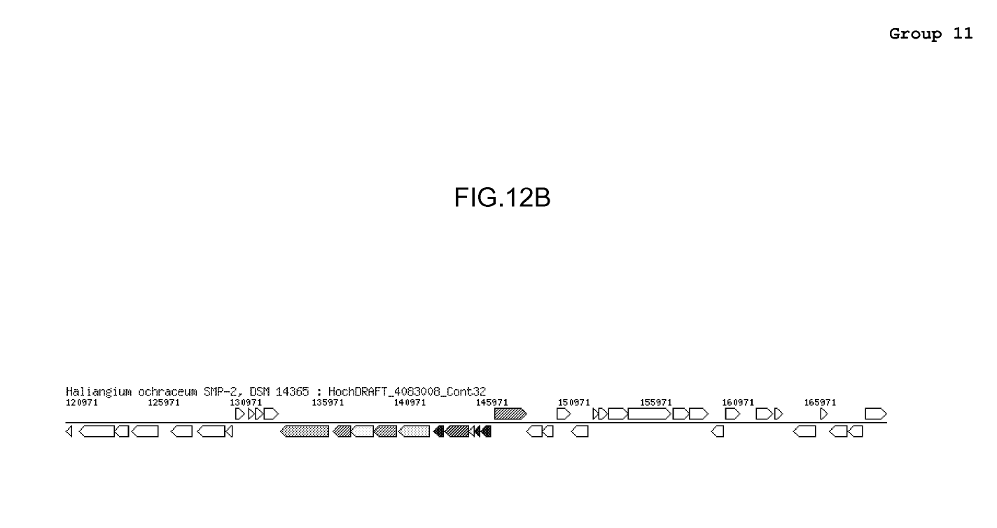

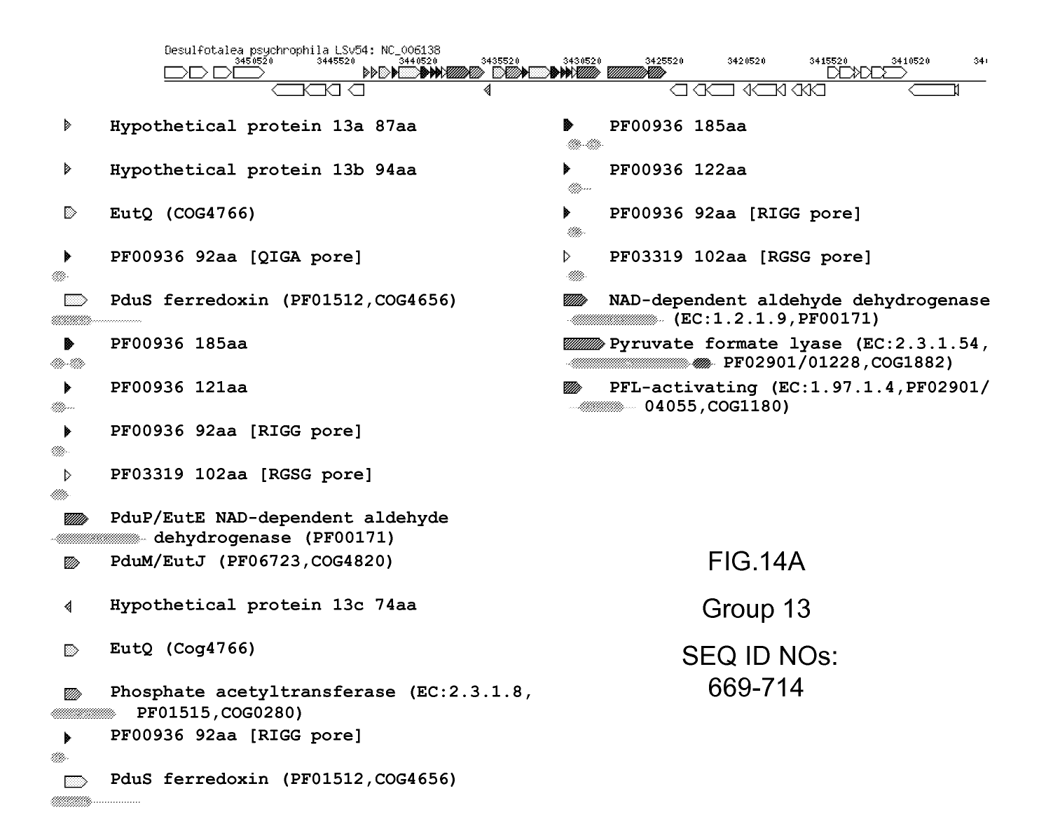

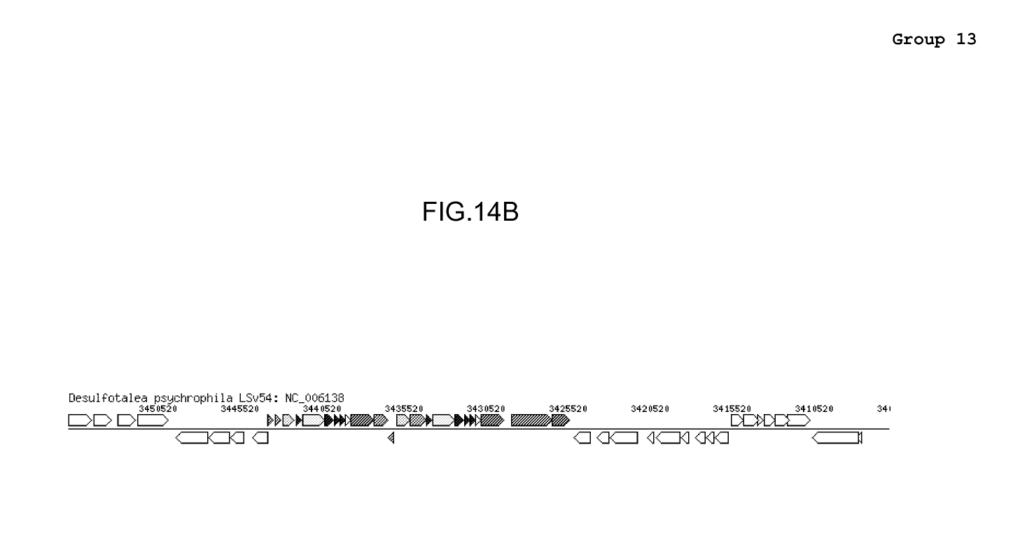

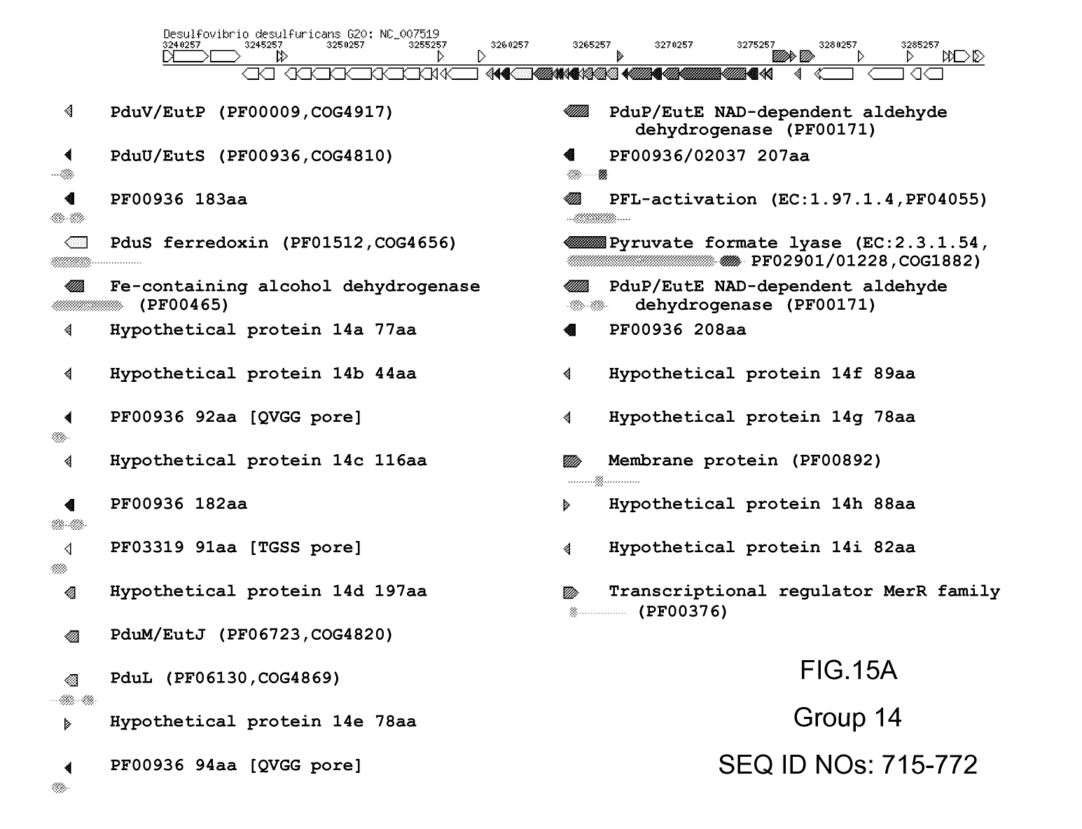

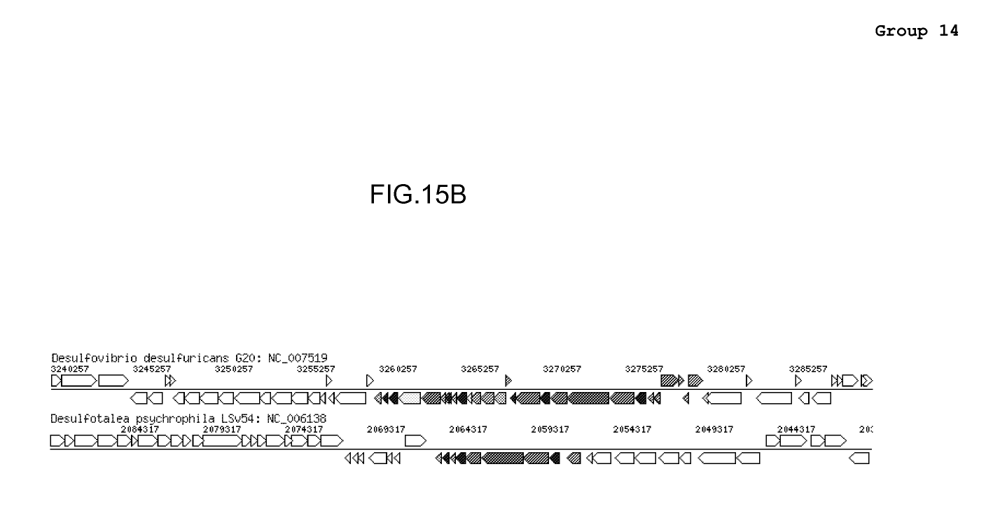

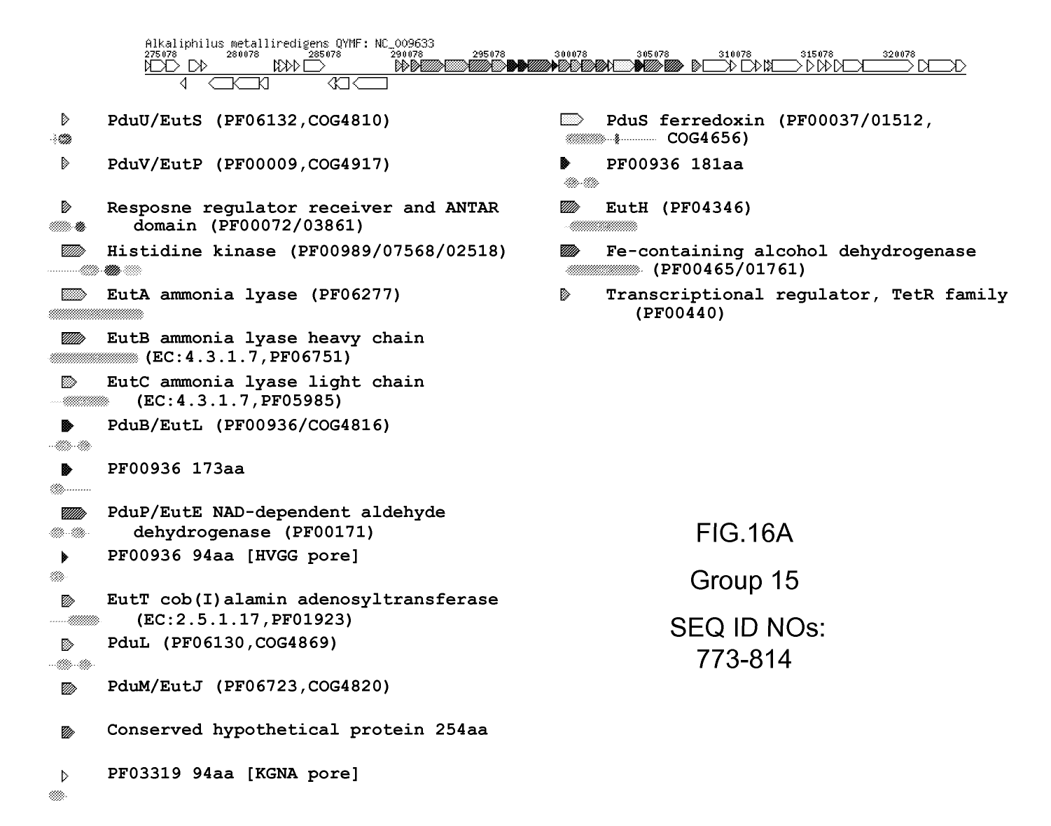

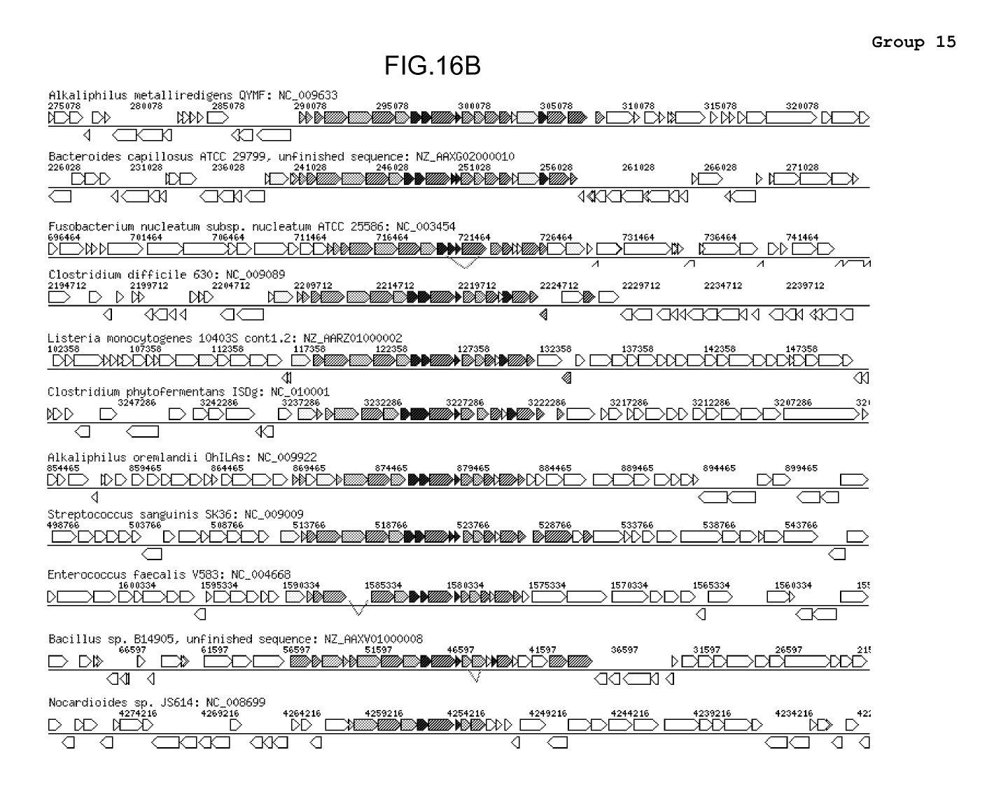

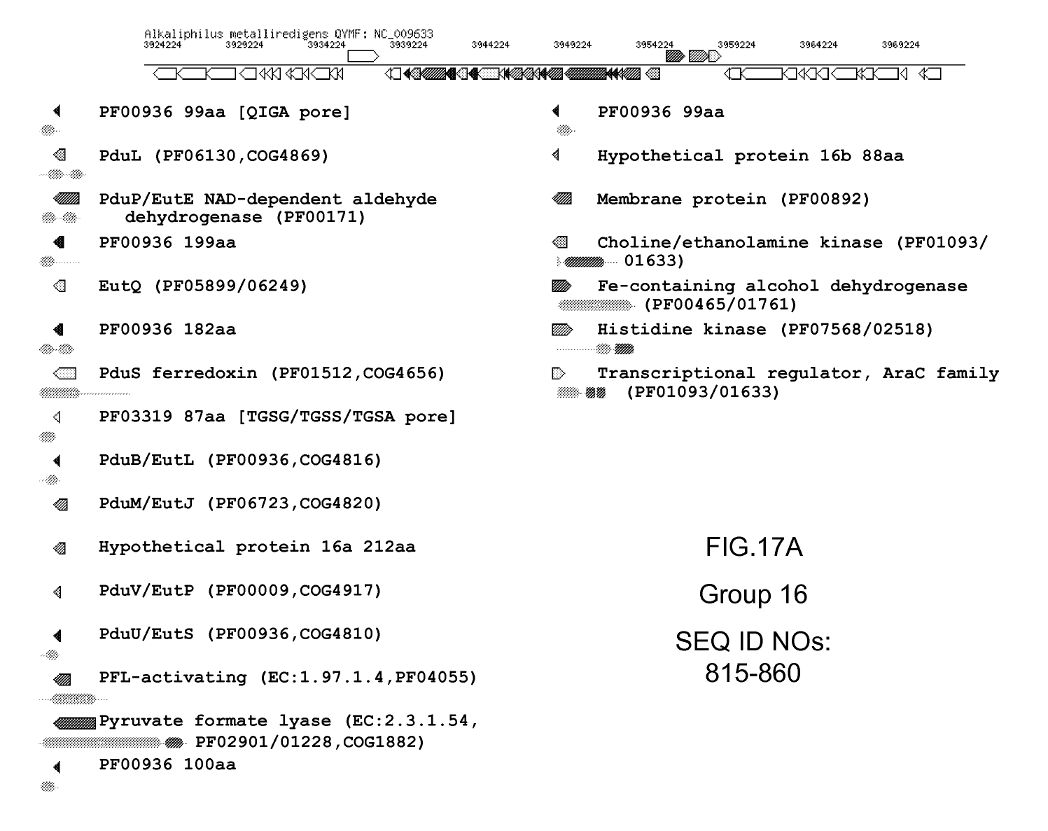

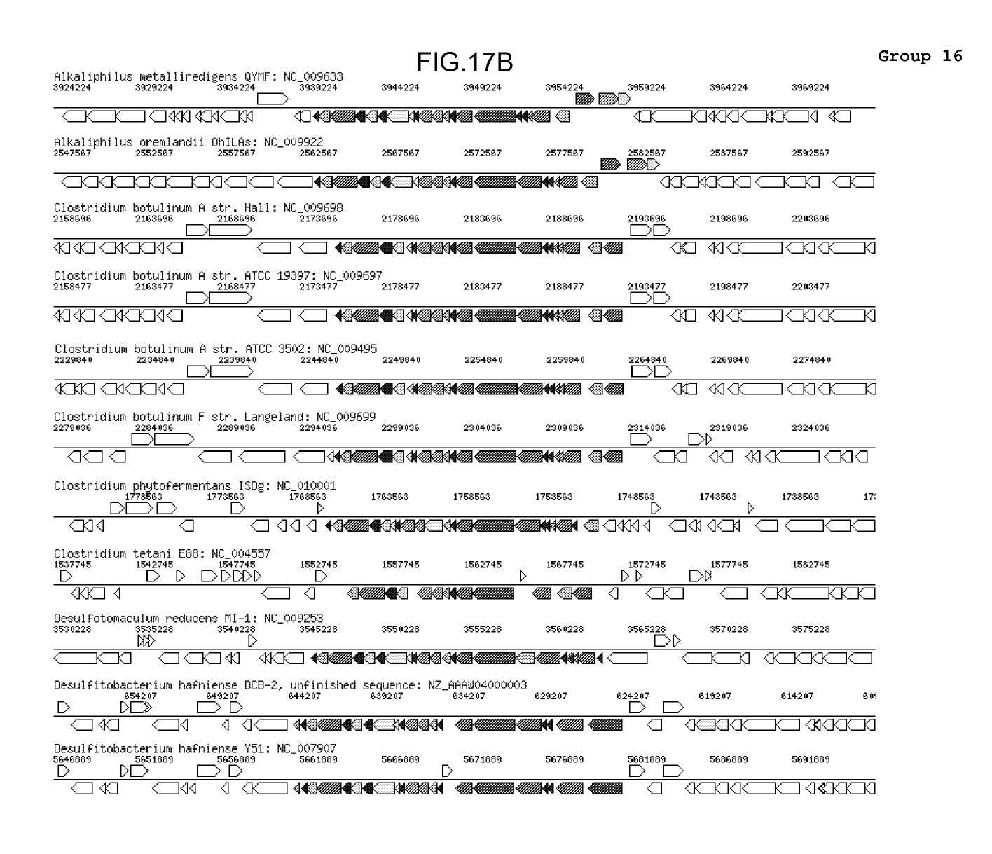

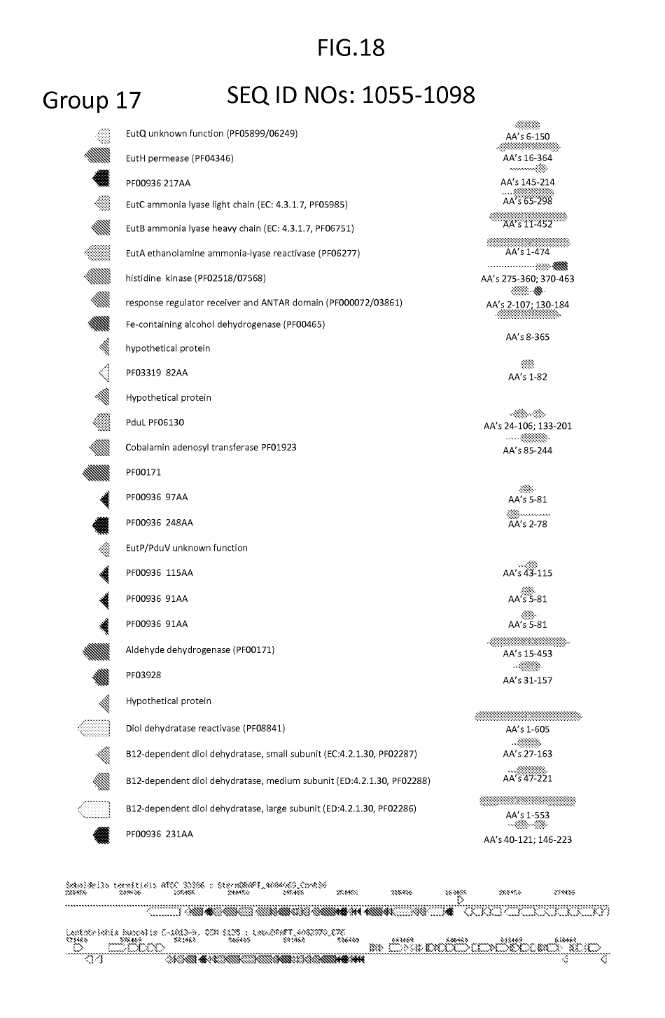

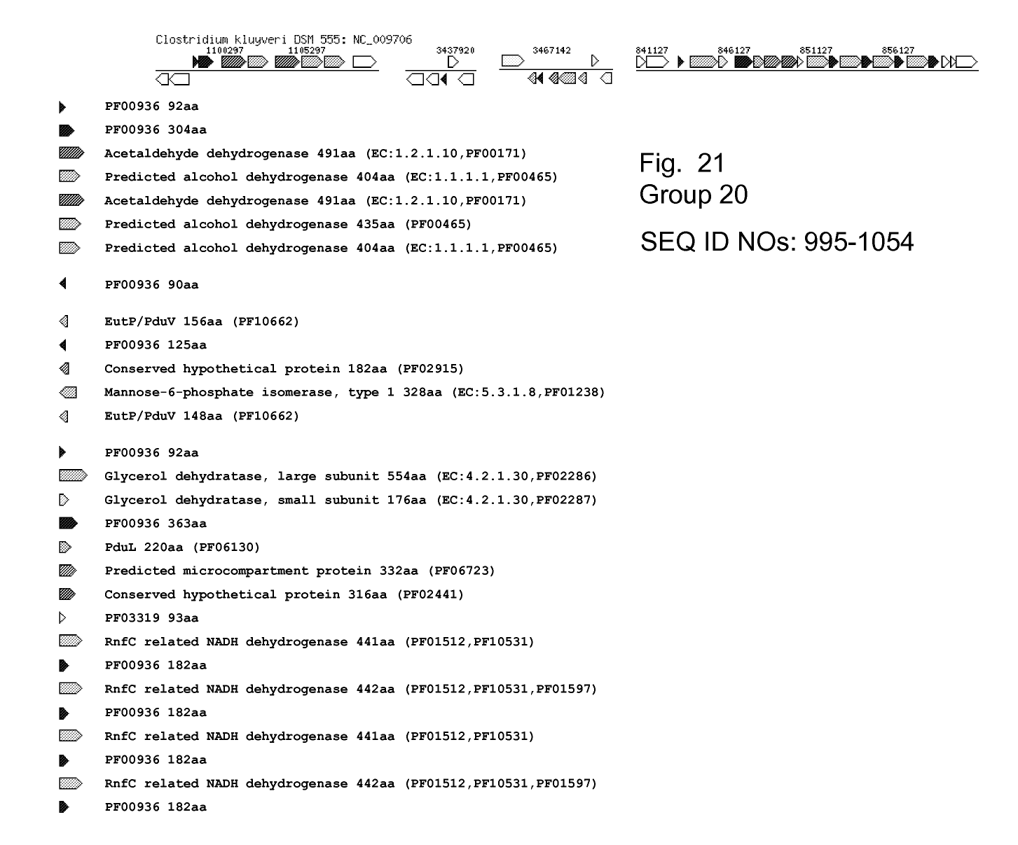

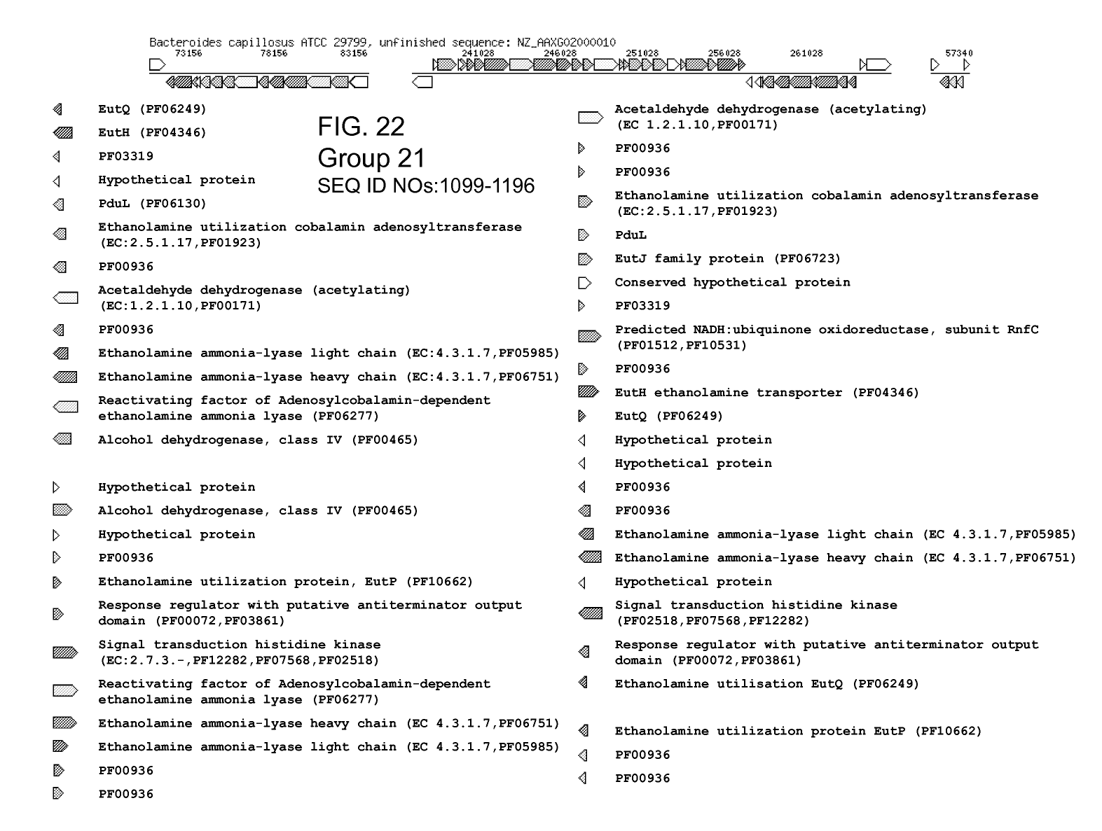

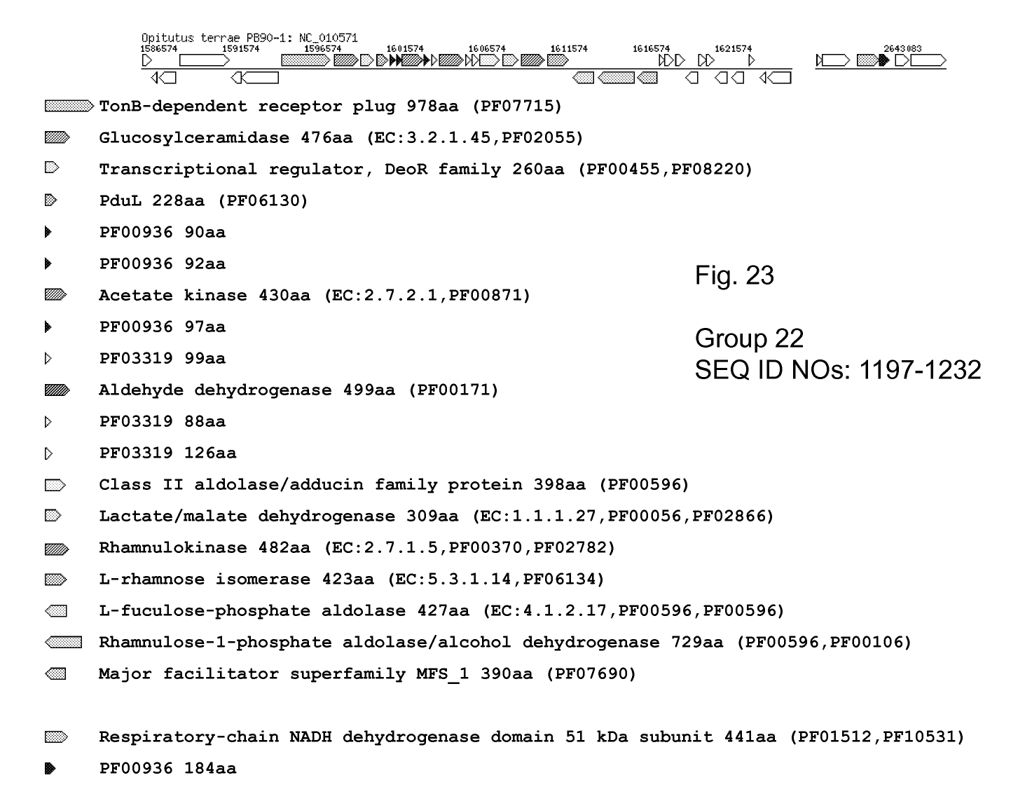

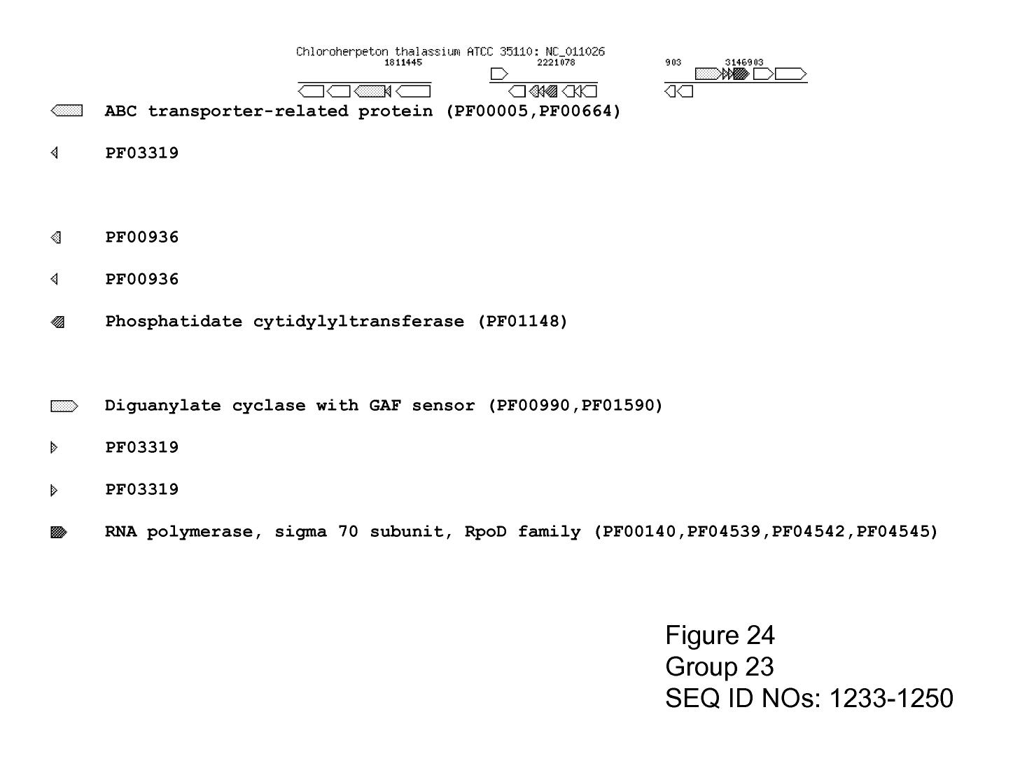

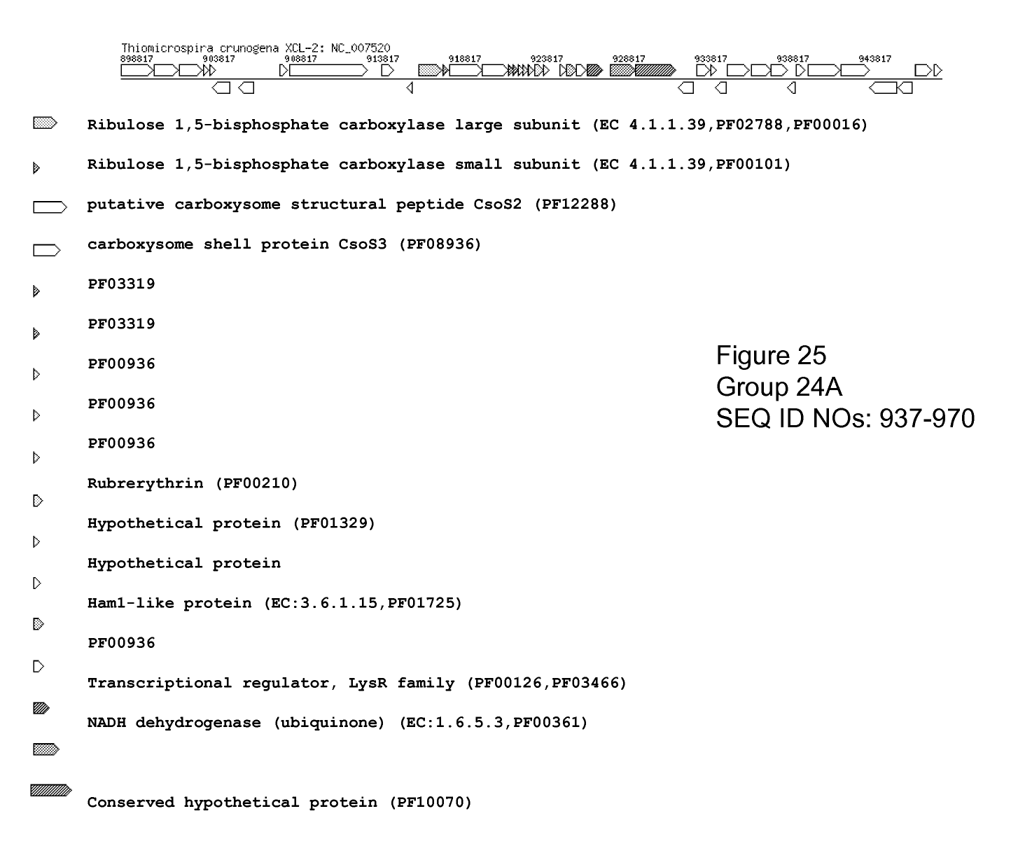

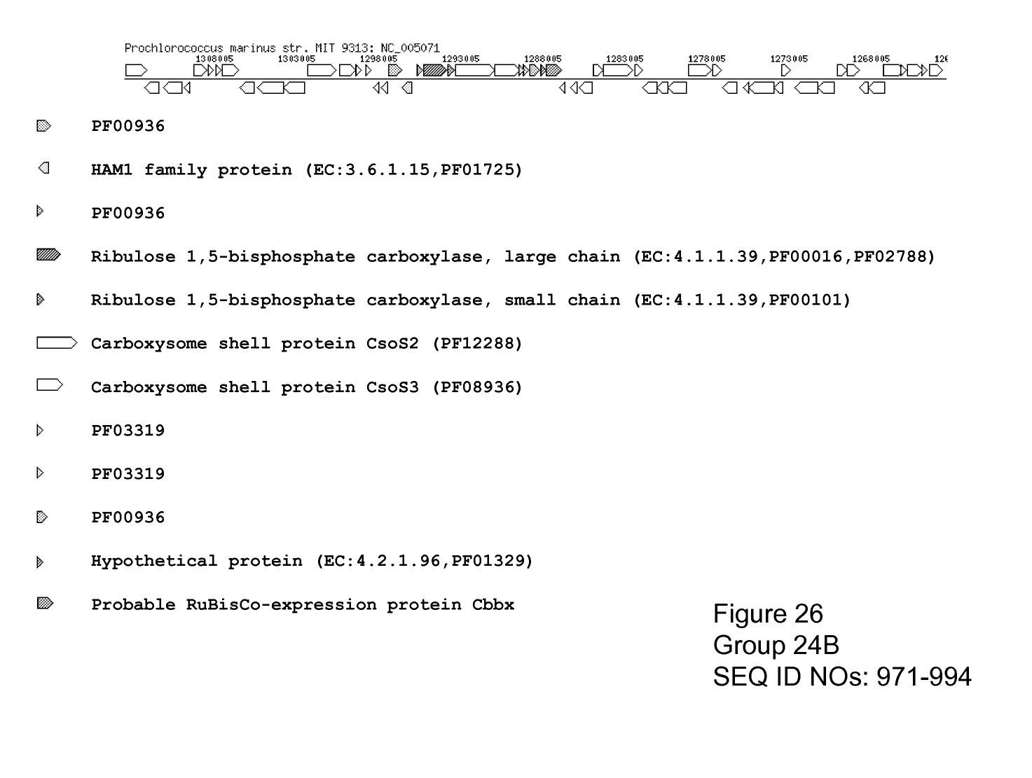

TABLE 3
Microcompartment Gene Cluster Groups
Group 1 (FIG. 2)
Aminotransferase (EC:2.6.1.-, PF00202, COG4992)-SEQ 1, 2
PduP/EutE NAD-dependent aldehyde dehydrogenase (PF00171, COG1012)-SEQ 3, 4
PF00936 201aa -SEQ 5, 6
Conserved hypothetical 01a--SEQ 7, 8
PF03319 84aa [QGSV pore]--SEQ 9, 10
PF00936 93aa [QVDG/EVDG pore]-SEQ 11, 12
Conserved hypothetical 01b-SEQ 13, 14
Aminoglycoside phosphotransferase (EC:5.4.2.1, PF01636)-SEQ 15, 16
Short-chain dehydrogenase/reductase (EC:1.1.1.100, PF00106)-SEQ 17, 18
Transcriptional regulator GntR family (PF00392/07702)-SEQ 19, 20
Group 2 (FIG. 3)
Pyruvate formate-lyase (PF01228/02901, COG1882) SEQ 69, 70
Pyruvate-formate lyase-activating enzyme (COG1180) SEQ 71, 72
L-fuculose phosphate aldolase (PF00596, COG0235) SEQ 73, 74
NAD-dependent aldehyde dehydrogenase (PF00171, COG1012), SEQ 75, 76
Threonine/Zn-dependent dehydrogenase dehydrogenases (PF00107/08240, SEQ 77, 78
PF00936 92aa SEQ 79, 80
PF00936 105aa SEQ 81, 82
PF00936 98aa SEQ 83, 84
PF00936 104aa SEQ 85, 86
Propanediol utilization protein (PF06130, COG4869) SEQ 87, 88
PF00936 88aa SEQ 89, 90
Electron transport complex protein RnfC (PF01512, COG4656) SEQ 91, 92
PF00936 182aa SEQ 93, 94
ABC-type cobalamin Fe3+-siderophores transport system (PF00455/08820/08279, COG1349)
SEQ 95, 96
Fe-containing alcohol dehydrogenase (PF00465, COG1454) SEQ 97, 98
Group 3 (FIG. 4)
Glutamate formiminotransferase (EC:2.1.2.5, PF07387/02971) SEQ 99, 100
Formate-tetrahydrofolate ligase (EC:6.3.4.3, PF01268) SEQ 101, 102
Allantoinase, dihydropyrimidinase (EC:3.5.2.2, PF01979, COG0044) SEQ 103, 104
Isochorismatase hydrolase (EC:3.5.1.19 PF00857, COG1335) SEQ 105, 106
3-octaprenyl-4-hydroxybenoate carboxylase (EC:4.1.1.-, PF02441) SEQ 107, 108
3-polyprenyl-4-hydroxybenoate decarboxylase (EC:4.1.1.-, PF01977) SEQ 109, 110
PF00936 91aa [YVGS pore] SEQ 111, 112
Hypothetical protein 3c SEQ 113, 114
PF00936 125aa SEQ 115, 116
PF00936 91aa [KIGF pore] SEQ 117, 118
Hypothetical protein 3b SEQ 119, 120
PF03319 90/96aa [RGTA/MGTA por] SEQ 121, 122
Adenine deaminase (EC:3.5.4.2, COG1001) SEQ 123, 124
Xant/ur/vit C permease (PF00860 SEQ 125, 126
Amidohydrolase (EC:3.5.4.2, PF01979, COG0402) SEQ 127, 128
Molybdopterin dehydrogenase (EC:1.2.99.2, PF00941) - SEQ 129, 130
2Fe-2S feRdoxin (PF01799/00111, COG2080) SEQ 131, 132
Xanthine dehydrogenase (EC:1.17.1.4, PF01315/02738, COG1529) SEQ 133,
Aldehyde oxidase (EC:1.2.3.1, PF02738, COG1529) SEQ 135, 136
Adenine deaminase (EC:2.5.4.2, PF01979/07968) SEQ 137, 138
Conserved hypothetical protein 3a SEQ 139, 140
Molybdenum cofactor biosynthesis COG2068)SEQ 141, 142
Group 4 (FIG. 5)
Fe-containing alcohol dehydrogenase (PF00465/01761)
PduL (PF06130, COG04869)
PduM/EutJ (COG4820)
Flavoprotein (PF02441)
PF00319 89aa [TGSS pore]
PduO (PF03928, COG3193)
Acetate kinase (EC:2.7.2.1, PF00871, COG0282)
PF00936 93aa [KIGS pore]
PduB/EutL (PF00936, COG4816)
PduP/EutE NAD-dependent aldehyde
Hypothetical protein 04a
Protease/amidase (PF01965, COG0693)
Pyruvate formate lyase (EC:2.3.1.54, PF02901/
PFL-activating (EC:1.97.1.4, PF04055, COG1180)
Conserved hypothetical protein
Putative maturase-related protein 179aa (COG3344)
Putative maturase-related protein 173aa (COG3344)
Hypothetical protein 04b/106aa, 04c/44aa, 04d/62aa
Histidine kinase (EC:2.7.3.-, PF06580/02150, COG3275)
Transcriptional regulator, AraC family(PF00165/00072, COG2207/2204)
Methionine adenosyltransferase (EC:2.5.1.6, PF00438/2772/2773)
PF00936 213/182aa
Superoxide response regulon transcriptional activator (PF00165, COG2207)
Transcriptional regulator, TetR family (PF00440)
Cation/multidrug efflux pump protein (COG0841)
Transposase InsC for insertion (PF01527,
Group 5 (FIG. 6)
Pyruvate formate lyase (EC:2.3.1.54,]-SEQ 269, 270
Fe-containing alcohol dehydrogenase (PF00465)-SEQ 267, 268
2 PF00936 97aa [KIGS pore]--SEQ 263, 264 AND 265, 266
PduB/EutL (PF00936, COG4816)-SEQ 261, 262
PF00936 93aa [KIGS pore]-SEQ 259, 260
PduL (PF06130, COG4869)SEQ 257, 258
PduM/EutJ (COG4820)-SEQ 255, 256
Flavoprotein (PF02441)-SEQ 253, 254
PF03319 89aa [CGSA pore]-SEQ 251, 252
PduO (PF03928, COG3193)-SEQ 249, 250
PduP/EutE NAD-dependent aldehyde dehydrogenase (PF00171)-SEQ 247, 248
Hypothetical protein 99aa (partial PF00936)-SEQ 245, 246
PFL-activating (EC:1.97.1.4, PF04055, COG1180)-SEQ 243, 244
Methionine adenosyltransferase (EC:2.5.1.6, PF00438/02772/02773)-SEQ 241, 242
Histidine kinase (EC:2.7.3.-, PF06580/02518)-SEQ 239, 240
Transcriptional regulator, AraC family (PF00072/00165, COG4753) SEQ 237, 238
Acetate kinase (EC:2.7.2.1, PF00871, COG0282)--SEQ 235, 236
Group 6 (FIG. 7)
Transposase Ins1 (PF03811, COG03677)
Transposase IS4 (PF01609)
PTS system, mannose/fructose/sorbose IIB subunit (PF03830)
PTS system, mannose/fructose/sorbose IIC subunit (PF03609)
PTS system, mannose/fructose/sorbose IID subunit (PF03613)
PF00936 100aa [KIGS pore]
PduB/EutL (PF00936, COG4816)
Pyruvate formate lyase (EC:2.3.1.54, PF02901/01228, COG1882)
PFL-activating (EC:1.97.1.4, PF00037/04055, COG1180)
PduF (PF00230, COG0580)
PF00936 96aa [KIGS pore]
PF00936 288aa
PduL (PF06130, COG4869)
PduM/EutJ (COG4820)
PF03319 92aa [RGSS pore]
PduO (PF03928, COG3193)
PduP/EutE NAD-dependent aldehyde dehydrogenase (PF00171)
Fe-containing alcohol dehydrogenase (PF00465/01761)
Transposase IS204/IS1001/IS1096/IS1165 (PF01610)
Lipoprotein signal peptidase (EC:3.4.23.36, PF01252, COG0597)
Cation efflux system permease (COG1230)
Transcriptional regulator, MerR family (PF00376/01381/07883)
Group 7 (FIG. 8)
Transposase IS3 (PF01527)
Integrase, catalytic region (PF00665)
Transcriptional regulator, TetR family(PF00440)
Transcriptional regulator, C-terminal (PF00486)
Hypothetical protein 07a
Hypothetical protein 07b
PF00936 92aa [NIGS pore]
PF00936 94aa [NIGS pore]
PF00936 92aa [NIGS pore]
PduP/EutE NAD-dependent aldehyde Dehydrogenase (PF00171)
PF03319 85aa [EYFA pore]
Fe-containing alcohol dehydrogenase PF00465/01761
Pyruvate formate lyase (EC:2.3.1.54, PF02901/01228, COG1882)
PFL-activating (EC:1.97.1.4, PF0037/04055, COG1180)
PF00936 150aa
PduL (PF06130, COG4869)
Hypothetical protein 07c
Multi-drug resistance protein (FP00893, COG2076)
Multi-drug resistance protein (FP00893, COG2076)
D-serine deaminase activator (PF00126/03466)
H+/gluconate symporter, GntP family(PF02447/03600, COG2610)
D-serine dehydratase (EC:4.3.1.18, PF00291, COG3048)
Group 8 (FIG. 9)
Acetyl-CoA C-acyltransferase (EC:2.3.1.16)
PF00936 93aa [KIGS pore]
PduB/EutL (PF00936, COG4816)
Glycerol dehydratase, large subunit (EC:4.2.1.30, PF02286, COG4909)
Glycerol dehydratase, medium subunit (EC:4.2.1.30, PF02288)
Glycerol dehydratase, small subunit (EC:4.2.1.30, PF02287, COG4910)
Putative glycerol dehydratase large subunit (EC:4.2.1.30, PF08841)
Hypothetical protein 121aa
PF00936 181aa
PduL (PF06130, COG4869)
PduM/EutJ (PF06723, COG4820)
Flavoprotein (PF02441)
ATP:cob(I)alamin adenosyltransferase (EC:2.5.1.17, PF01923/03928)
Protein of unknown function (PF03928)
PduP/EutE NAD-dependent aldehyde dehydrogenase (PF00171)
PduS ferredoxin (PF01512, COG4656)
PF00936 183aa
Hypothetical protein
Butyrate kinase (EC:2.7.2.7, PF00871)
Acetate kinase (EC:2.7.2.1, PF00871)
Fe-containing alcohol dehydrogenase (PF00465)
ATPase-like protein
Hypothetical protein
Membrane protein (PF04020)
Transcriptional regulator, TetR family (PF00440)
Group 9 (FIG. 10)
Malate dehydrogenase (EC:1.1.1.37, PF00056/02866, COG0039)-559, 560
L-fuculose-phosphate aldolase (EC:4.1.2.17, PF00596, COG0235)-SEQ 557, 558
PF03319 96/101/93aa [EGGE/EGPE/EGAE pore]-SEQ 555, 556
Hypothetical protein 09a 222aa-SEQ 553, 554
PF00319 86/95/86aa [SDGE/SETG/SDGA pore]-SEQ 551, 552
Aldehyde dehydrogenase (EC:1.2.1.10, PF00171, COG1012)-SEQ 549, 550
PF03319 146/130/85aa [QGSS/QGSS/SDGA pore]-SEQ 547, 548
Hypothetical protein 09b 44aa-SEQ 545, 546
Acetate kinase (EC:2.7.2.1, PF00871, COG0282)-SEQ 543, 544
PF00936 100aa [NIGG/KIGA/QIGG pore]-SEQ 541, 542
PF00936 100aa [KVGS/KIGA/KVGS pore]-SEQ 539, 540
PduL (PF06130, COG4869)-SEQ 537, 538
Transcriptional regulator, DeoR family (PF08220/00455, COG1349)-SEQ 535, 536
Group 10 (FIG. 11)
Sugar diacid utilization regulator (PF01590, COG3835)
Zn-containing alcohol dehydrogenase (PF08240/00107, COG1063)
Malate dehydrogenase (EC:1.1.1.37, PF00056/02866, COG0039)
PF00936 205aa
PduM/EutJ (PF06723, COG4820)
PduP/EutE NAD-dependent aldehyde dehydrogenase (PF00171)
PF00936 105aa [QIGG pore]
PF03319 103aa [LGSA pore]
Hypothetical protein 10a 332aa
Phosphatase-like protein (EC:3.1.3.18, PF00702)
Hypothetical protein 10b 104aa
Pyruvate phosphate dikinase (EC:2.7.9.1, PF01326/00391, COG0574)
PF00936 100aa [QPGG pore]
Hypothetical protein 10c 223aa
TonB-dependent outer membrane cobalamin receptor (PF07715/00593)
Cobrinic acid a,c-diamide synthase (EC:6.3.5.9, PF01656/07685)
Adenosyl cobinamide kinase (EC:2.7.1.156, (EC:2.7.1.156, PF02283)
Iron(III) dicitrate-binding protein (PF01497)
Iron ABC transporter, permease protein (EC:3.6.3.33, PF01032)
Iron ABC transporter ATP-binding protein (EC:3.6.3.34, PF00005)
Cob(I)alamin adenosyltransferase (EC:2.5.1.17, PF02572)
Cobalamin biosynthesis (EC:6.3.1.10, PF03186)
Cobyric acid decarboxylase (EC:2.6.1.9, PF00155)
Cobyric acid synthase (EC:6.3.5.10, PF01656/07685)
Group 11 (FIG. 12)
PAS domain S-box (PF00072/00512/00785/00989/02518)
Putative homoserine kinase type II (PF01636)
FKBP-type peptidyl-prolyl cis-trans isomerase (EC:5.2.1.8, PF00254)
Xaa-pro aminopeptidase (EC:3.4.13.9, PF00557)
Serine/threonine-protein kinase (EC:2.7.11.1, PF00069)
PF00936 205aa
PduP/EutE NAD-dependent aldehyde dehydrogenase (PF00171)
PF03319 96aa [SGSS pore]
PF00936 84aa [KTGG pore]
PF00936 212aa
Peptidase C11 (PF03415)
Group 12 (FIG. 13A and 13B)
Transcriptional regulator, LysR family (PF00126/03466)
PF00936 260aa
CcmN 248aa
CcmM (EC:4.2.1.1, PF00101)
PF03319 101aa [VVGA pore]
PF00946 114aa [KIGS pore]
PF00936 114aa [KIGS pore]
NADH dehydrogenase subunit L (EC:1.6.99.5, PF00361)
NADH dehydrogenase subunit M (EC:1.6.99.5, PF00361)
Group 12A (FIG. 13C and 13D)
PF00936 258aa
CcmN 304aa
Protein tyrosine phosphatase (COG0394)
CcmM 672aa
PF03319 100aa [RGSA pore]
PF00946 112aa [KIGS pore]
PF00936 103aa [KIGS pore]
Group 13 (FIG. 14)
Hypothetical protein 13a 87aa
Hypothetical protein 13b 94aa
EutQ (COG4766)
PF00936 92aa [QIGA pore]
PduS ferredoxin (PF01512, COG4656)
PF00936 185aa
PF00936 121aa
PF00936 92aa [RIGG pore]
PF03319 102aa [RGSG pore]
PduP/EutE NAD-dependent aldehyde dehydrogenase (PF00171)
PduM/EutJ (PF06723, COG4820)
Hypothetical protein 13c 74aa
EutQ (Cog4766)
Phosphate acetyltransferase (EC:2.3.1.8, PF01515, COG0280)
PF00936 92aa [RIGG pore]
PduS ferredoxin (PF01512, COG4656)
PF00936 185aa
PF00936 122aa
PF00936 92aa [RIGG pore]
PF03319 102aa [RGSG pore]
NAD-dependent aldehyde dehydrogenase (EC:1.2.1.9, PF00171)
Pyruvate formate lyase (EC:2.3.1.54, PF02901/01228, COG1882)
PFL-activating (EC:1.97.1.4, PF02901/04055, COG1180)
Group 14 (FIG. 15)
PduV/EutP (PF00009, COG4917)
PduU/EutS (PF00936, COG4810)
PF00936 183aa
PduS ferredoxin (PF01512, COG4656)
Fe-containing alcohol dehydrogenase (PF00465)
Hypothetical protein 14a 77aa
Hypothetical protein 14b 44aa
PF00936 92aa [QVGG pore]
Hypothetical protein 14c 116aa
PF00936 182aa
PF03319 91aa [TGSS pore]
Hypothetical protein 14d 197aa
PduM/EutJ (PF06723, COG4820)
PduL (PF06130, COG4869)
Hypothetical protein 14e 78aa
PF00936 94aa [QVGG pore]
PduP/EutE NAD-dependent aldehyde dehydrogenase (PF00171)
PF00936/02037 207aa
PFL-activation (EC:1.97.1.4, PF04055)
Pyruvate formate lyase (EC:2.3.1.54, PF02901/01228, COG1882)
PduP/EutE NAD-dependent aldehyde dehydrogenase (PF00171)
PF00936 208aa
Hypothetical protein 14f 89aa
Hypothetical protein 14g 78aa
Membrane protein (PF00892)
Hypothetical protein 14h 88aa
Hypothetical protein 14i 82aa
Transcriptional regulator MerR family (PF00376)
Group 15 (FIG. 16)
PduU/EutS (PF06132, COG4810)
PduV/EutP (PF00009, COG4917)
Resposne regulator receiver and ANTAR domain (PF00072/03861)
Histidine kinase (PF00989/07568/02518)
EutA ammonia lyase (PF06277)
EutB ammonia lyase heavy chain (EC:4.3.1.7, PF06751)
EutC ammonia lyase light chain (EC:4.3.1.7, PF05985)
PduB/EutL (PF00936/COG4816)
PF00936 173aa
PduP/EutE NAD-dependent aldehyde dehydrogenase (PF00171)
PF00936 94aa [HVGG pore]
EutT cob(I)alamin adenosyltransferase (EC:2.5.1.17, PF01923)
PduL (PF06130, COG4869)
PduM/EutJ (PF06723, COG4820)
Conserved hypothetical protein 254aa
PF03319 94aa [KGNA pore]
PduS ferredoxin (PF00037/01512, COG4656)
PF00936 181aa
EutH (PF04346)
Fe-containing alcohol dehydrogenase (PF00465/01761)
Transcriptional regulator, TetR family (PF00440)
Group 16 (FIG. 17)
PF00936 99aa [QIGA pore]
PduL (PF06130, COG4869)
PduP/EutE NAD-dependent aldehyde dehydrogenase (PF00171)
PF00936 199aa
EutQ (PF05899/06249)
PF00936 182aa
PduS ferredoxin (PF01512, COG4656)
PF03319 87aa [TGSG/TGSS/TGSA pore]
PduB/EutL (PF00936, COG4816)
PduM/EutJ (PF06723, COG4820)
Hypothetical protein 16a 212aa
PduV/EutP (PF00009, COG4917)
PduU/EutS (PF00936, COG4810)
PFL-activating (EC:1.97.1.4, PF04055)
Pyruvate formate lyase (EC:2.3.1.54, PF02901/01228, COG1882)
PF00936 100aa
PF00936 99aa
Hypothetical protein 16b 88aa
Membrane protein (PF00892)
Choline/ethanolamine kinase (PF01093/01633)
Fe-containing alcohol dehydrogenase (PF00465/01761)
Histidine kinase (PF07568/02518)
Transcriptional regulator, AraC family (PF01093/01633)
Group 17 (FIG. 18)
EutQ unknown function (PF05899/06249)
EutH permease (PF04346)
PF00936217aa
EutC ammonia lyase light chain (EC:4.3.1.7, PF05985)
EutB ammonia lyase heavy chain (EC:4.3.1.7, PF06751)
EutA ethanolamine ammonia-lyase reactivase (PF06277)
Histidine kinase (PF07568/02518)
Response regulator receiver and NTAR domain (PF00072/03861)
Fe-containing alcohol dehydrogenase (PF00465)
Hypothetical protein
PF03319 82aa
Hypothetical protein
PduL PF06130
Cobalamin adenonsyl transferase PF01923
PF00171
PF00936 97aa
PF00936 248aa
EutP/PduV unknown function
PF00936 115aa
PF00936 91aa
PF00936 91aa
Aldehyde dehydrogenase PF00171)
PF03928
Hypothetical protein
Diol dehydratase reactivase (PF08841)
B12-dependent diol dehydratase, small subunit (E:4.2.1.30, PF02287)
B12-dependent diol dehydratase, medium subunit (E:4.2.1.30, PF02288)
B12-dependent diol dehydratase, large subunit (E:4.2.1.30, PF02286)
PF00936 231 aa
Group 18 (FIG. 19)
PF00936 94aa [KIGS pore]
PduB/EutL (PF00936, COG4816)
PduC B12-dependent diol dehydratase, large subunit (EC:4.2.1.30, PF08841)
PduC B12-dependent diol dehydratase, medium subunit (EC:4.2.1.30, PF02288)
PduC B12-dependent diol dehydratase, small subunit (EC:4.2.1.30, PF002287)
PduG diol dehydratase reactivase
PduH diol dehydratase reactivase
PF00936 91aa [KIGS pore]
PF00936 160aa
PduL phosphotransacylase (PF06130, COG4869)
PduM/EutJ possible chaperone (PF06723, COG4820)
PF03319 91aa [GGSS pore]
PduO adenosyl transferase (PF01923/03928, COG3193)
PduP/EutE propionaldehyde dehydrogenase (PF00171)
PduQ/EutG propanol dehydrogenase (PF00465)
PduS cobalamin reductase (PF01512 COG4656)
PF00936 184aa
PduU/EutS (PF00936, COG4810)
PduV/EutP (PF00009, COG4917)
PduW acetate kinase (EC:2.7.2.1,
PduX threonine kinase (PF00288/08544)
Group 19 (FIG. 20)
EutS/PduU (PF00936, COG4810) (SEQ ID NOs: 905, 906)
EutP/PduV unknown function (SEQ ID NOs: 907, 908)
EutQ unknown function (PF05899/06249) (SEQ ID NOs: 909, 910)
EutT corrinoid adenosyltransferase, cobalamin recycling (EC:2.5.1.17, PF01923) (SEQ ID
NOs: 911, 912)
EutD phosphotransacetylase (PF01515) (SEQ ID NOs: 913, 914)
EutM (PF00936 96aa [QIGG pore]) (SEQ ID NOs: 915, 916)
EutN (PF03319 99aa [SGSS pore]) (SEQ ID NOs: 917, 918)
EutE/PduP aldehyde dehydrogenase (PF00171) (SEQ ID Nos: 919, 920)
EutJ/PduM possible chaperone (PF06723, COG4820) (SEQ ID Nos : 921, 922)
EutG/PduQ alcohol dehydrogenase (PF00465) (SEQ ID NOs: 923, 924)
EutH permease (PF04346) (SEQ ID NOs: 925, 926)
EutA ethanolamine ammonia-lyase reactivase (PF06277) (SEQ ID NOs: 927, 928)
EutB ammonia lyase heavy chain (EC:4.3.1.7, PF06751) (SEQ ID NOs: 929, 930)
EutC ammonia lyase light chain (EC:4.3.1.7, PF05985) (SEQ ID NOs: 931, 932)
EutL/PduL (PF00936, COG4816) 219aa (SEQ ID NOs: 933, 934)
EutK (PF03319) 164aa (SEQ ID NOs: 935, 936)
EutR transcriptional activator, AraC family (SEQ ID NOs: 903, 904)
Group 20 (FIG. 21)
PF00936 92aa
PF00936 304aa
Acetaldehyde dehydrogenase 491aa (EC:1.2.1.10, PF00171)
Predicted alcohol dehydrogenase 404aa (EC:1.1.1.1, PF00465)
Acetaldehyde dehydrogenase 491aa (EC:1.2.1.10, PF00171)
Predicted alcohol dehydrogenase 435aa (PF00465)
Predicted alcohol dehydrogenase 404aa (EC:1.1.1.1, PF00465)
PF00936 90aa
EutP/PduV 156aa (PF10662)
PF00936 125aa
Conserved hypothetical protein 182aa (PF02915)
Mannose-6-phosphate isomerase, type 1 328aa (EC:5.3.1.8, PF01238)
EutP/PduV 148aa (PF10662)
PF00936 92aa
Glycerol dehydratase, large subunit 554aa (EC:4.2.1.30, PF02286)
Glycerol dehydratase, small subunit 176aa (EC:4.2.1.30, PF02287)
PF00936 363aa
PduL 220aa (PF06130)
Predicted microcompartment protein 332aa (PF06723)
Conserved hypothetical protein 316aa (PF02441)
PF03319 93aa
RnfC related NADH dehydrogenase 441′aa (PF01512, PF10531)
PF00936 182aa
RnfC related NADH dehydrogenase 442aa (PF01512, PF10531, PF01597)
PF00936 182aa
RnfC related NADH dehydrogenase 441aa (PF01512, PF10531)
PF00936 182aa
RnfC related NADH dehydrogenase 442aa (PF01512, PF10531, PF01597)
PF00936 182aa
Group 21 (FIG. 22)
EutQ (PF06249)
EutH (PF04346)
PF03319
Hypothetical protein
PduL (PF06130)
Ethanolamine utilization cobalamin adenosyltransferase (EC:2.5.1.17, PF01923)
PF00936
Acetaldehyde dehydrogenase (acetylating) (EC:1.2.1.10, PF00171)
PF00936
Ethanolamine ammonia-lyase light chain (EC:4.3.1.7, PF05985)
Ethanolamine ammonia-lyase heavy chain (EC:4.3.1.7, PF06751)
Reactivating factor of Adenosylcobalamin-dependent ethanolamine ammonia lyase (PF06277)
Alcohol dehydrogenase, class IV (PF00465)
Hypothetical protein
Alcohol dehydrogenase, class IV (PF00465)
Hypothetical protein
PF00936
Ethanolamine utilization protein, EutP (PF10662)
Response regulator with putative antiterminator output domain (PF00072, PF03861)
Signal transduction histidine kinase
(EC:2.7.3.-, PF12282, PF07568, PF02518)
Reactivating factor of Adenosylcobalamin-dependent ethanolamine ammonia lyase (PF06277)
Ethanolamine ammonia-lyase heavy chain (EC 4.3.1.7, PF06751)
Ethanolamine ammonia-lyase light chain (EC 4.3.1.7, PF05985)
PF00936
PF00936
Acetaldehyde dehydrogenase (acetylating)
(EC 1.2.1.10, PF00171)
PF00936
PF00936
Ethanolamine utilization cobalamin adenosyltransferase (EC:2.5.1.17, PF01923)
PduL
EutJ family protein (PF06723)
Conserved hypothetical protein
PF03319
Predicted NADH:ubiquinone oxidoreductase, subunit RnfC (PF01512, PF10531)
PF00936
EutH ethanolamine transporter (PF04346)
EutQ (PF06249)
Hypothetical protein
Hypothetical protein
PF00936
PF00936
Ethanolamine ammonia-lyase light chain (EC 4.3.1.7, PF05985)
Ethanolamine ammonia-lyase heavy chain (EC 4.3.1.7, PF06751)
Hypothetical protein
Signal transduction histidine kinase (PF02518, PF07568, PF12282)
Response regulator with putative antiterminator output domain (PF00072, PF03861)
Ethanolamine utilisation EutQ (PF06249)
Ethanolamine utilization protein EutP (PF10662)
PF00936
PF00936
Group 22 (FIG. 23)
TonB-dependent receptor plug 978aa (PF07715)
Glucosylceramidase 476aa (EC:3.2.1.45, PF02055)
Transcriptional regulator, DeoR family 260aa (PF00455, PF08220)
PduL 228aa (PF06130)
PF00936 90aa
PF00936 92aa
Acetate kinase 430aa (EC:2.7.2.1, PF00871)
PF00936 97aa
PF03319 99aa
Aldehyde dehydrogenase 499aa (PF00171)
PF03319 88aa
PF03319 126aa
Class II aldolase/adducin family protein 398aa (PF00596)
Lactate/malate dehydrogenase 309aa (EC:1.1.1.27, PF00056, PF02866)
Rhamnulokinase 482aa (EC:2.7.1.5, PF00370, PF02782)
L-rhamnose isomerase 423aa (EC:5.3.1.14, PF06134)
L-fuculose-phosphate aldolase 427aa (EC:4.1.2.17, PF00596, PF00596)
Rhamnulose-1-phosphate aldolase/alcohol dehydrogenase 729aa (PF00596, PF00106)
Major facilitator superfamily MFS_1 390aa (PF07690)
Respiratory-chain NADH dehydrogenase domain 51 kDa subunit 441aa (PF01512, PF10531)
PF00936 184aa
Group 23 (FIG. 24)
ABC transporter-related protein (PF00005, PF00664)
PF03319
PF00936
PF00936
Phosphatidate cytidylyltransferase (PF01148)
Diguanylate cyclase with GAF sensor (PF00990, PF01590)
PF03319
PF03319
RNA polymerase, sigma 70 subunit, RpoD family (PF00140, PF04539, PF04542, PF04545)
Group 24A (FIG. 25)
Ribulose 1,5-bisphosphate carboxylase large subunit (EC 4.1.1.39, PF02788, PF00016)
Ribulose 1,5-bisphosphate carboxylase small subunit (EC 4.1.1.39, PF00101)
putative carboxysome structural peptide CsoS2 (PF12288)
carboxysome shell protein CsoS3 (PF08936)
PF03319
PF03319
PF00936
PF00936
PF00936
Rubrerythrin (PF00210)
Hypothetical protein (PF01329)
Hypothetical protein
Ham 1-like protein (EC:3.6.1.15, PF01725)
PF00936
Transcriptional regulator, LysR family (PF00126, PF03466)
NADH dehydrogenase (ubiquinone) (EC:1.6.5.3, PF00361)
Hypothetical protein
Conserved hypothetical protein (PF10070)
Group 24B (FIG. 26)
PF00936
HAM1 family protein (EC:3.6.1.15, PF01725)
PF00936
Ribulose 1,5-bisphosphate carboxylase, large chain (EC:4.1.1.39, PF00016, PF02788)
Ribulose 1,5-bisphosphate carboxylase, small chain (EC:4.1.1.39, PF00101)
Carboxysome shell protein CsoS2 (PF12288)
Carboxysome shell protein CsoS3 (PF08936)
PF03319
PF03319
PF00936
Hypothetical protein (EC:4.2.1.96, PF01329)
Probable RuBisCo-expression protein Cbbx

It is contemplated that other organisms other than those shown in the Figures as also containing the Group of genes, will be found. The other organisms shown in the Figures as falling into a particular group as having the same cluster of genes is not to be seen as a finite or limiting list of organisms that may be contained within any particular Group. It is further contemplated that new Groups will be found based on the presence of bacterial micrompartment genes (Pfam 00936 and or Pfam03319) in their genomes in association with other genes encoding other enzymatic or protein functions and those Groups may be added to the present microcompartment catalog.

### Applications for Bacterial Microcompartment Sequences and Groups

Compartments and their associated proteins and enzymes as listed in the Sequence Listing and the Figures find use in transforming plants, seeds, and plant products, algae, bacteria and archaea in a variety of ways as described below and in the following Examples.

To test if the protein products of the selected genes have activity (e.g., carbon fixation activity), cell-free protein synthesis can be used to translate the DNA sequence of each gene into protein.

In one embodiment, genes encoding a bacterial compartment are cloned into an appropriate plasmid under an inducible promoter, inserted into vector, and used to transform cells, such as E. coli, cyanobacteria, plants, algae, or other photosynthetic organisms. This system maintains the expression of the inserted gene silent unless an inducer molecule (e.g., IPTG) is added to the medium.

Bacterial colonies are allowed to grow after induction of gene expression. In one embodiment, the presently described genes, proteins and/or RNA described in SEQ ID NOS: 1-1268, and herein referred to as generally bacterial compartments or microcompartments, are contemplated for use in any of the applications herein described. When referring to the bacterial compartments or microcompartments, it is meant to include any number of proteins, shell proteins or enzymes (e.g., dehydrogenases, aldolases, lyases, etc.) that comprise or are encapsulated in the compartment.

In another embodiment, an expression vector comprising a nucleic acid sequence for a cluster of bacterial compartment genes, selected from any of the polynucleotide sequences in SEQ ID NOS:1-1268, is expressed in an organism by addition of an inducer molecule.

In some embodiments, expression cassettes comprising a promoter operably linked to a heterologous nucleotide sequence of the invention, i.e., any nucleotide sequence in SEQ ID NOS:1-1268, that encodes a microcompartment RNA or polypeptide are further provided. In another embodiment, the expression cassette comprising the sequences of genes of one of the Groups of Table 1. Thus in another embodiment, the cassette is selected from the following groups of sequences: SEQ ID NOS: 1-20, 21-44, 45-68, 69-98, 99-146, 147-176, 177-234, 235-270, 271-296, 297-342, 343-386, 387-436, 437-482, 483-534, 535-560, 561-608, 609-634, 635-652 and 1251-1260, 653-668 and 1261-1268, 669-714, 715-772, 773-814, 815-860, 1055-1098, 861-902, 903-936-, 937-970, 971-994, 995-1054, 1099-1196, 1197-1232, or 1233-1250.

In some embodiments as in some organisms, the BMC gene cluster in the expression cassette is interrupted by a gene encoded off the opposite strand (see for example, FIG. 26A, Group 24B, in Prochlorococcus marinus MIT 9313, the second gene in the Group). Such interruptions may be important in regulation and/or stoichiometry and can be employed. In other embodiments, there is intergenic spacing which can be roughly proportional to the gaps in between genes in the rest of the genome (see for example, in FIG. 13C, Group 12A proxy organism, Trichodesmium erythraeum for some reason, prefers a lot of space between all of its genes, not just in BMCs).

The expression cassettes of the invention find use in generating transformed prokaryotic, eukaryotic cells and microorganisms, plants, and plant cells. The expression cassette will include 5′ and 3′ regulatory sequences operably linked to a polynucleotide of the invention. “Operably linked” is intended to mean functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide that encodes a microcompartment RNA or polypeptide to be under the transcriptional regulation of the regulatory regions. The expression cassette may additionally contain selectable marker genes.

The expression cassette will include in the 5′-3′ direction of transcription, a transcriptional initiation region (i.e., a promoter), translational initiation region, a polynucleotide of the invention, a translational termination region and, optionally, a transcriptional termination region functional in the host organism. The regulatory regions (i.e., promoters, transcriptional regulatory regions, and translational termination regions) and/or the polynucleotide of the invention may be native/analogous to the host cell or to each other. Alternatively, the regulatory regions and/or the polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

Where appropriate, the polynucleotides may be optimized for increased expression in the transformed organism. For example, the polynucleotides can be synthesized using preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). The above list of selectable marker genes is not meant to be limiting. Any selectable marker gene can be used in the present invention.

Generally, it will be beneficial to express the genes from an inducible promoter.

In one embodiment, a eukaryote, such as a plant, transformed by the microcompartment RNA or polypeptides of the present invention is a plant (or an offspring thereof) which is regenerated on the basis of host plant cells transformed with the gene of the present invention located under the control of a suitable promoter capable of functioning in eukaryotic cells, or with the gene of the present invention integrated in a suitable vector. The transformed organism of the present invention can express, in its body, the microcompartment and enzymes or proteins for metabolizing activity according to the present invention.

The expression vector usable in the method of transforming plant cells with the gene of the present invention include pUC vectors (for example pUC118, pUC119), pBR vectors (for example pBR322), pBI vectors (for example pBI112, pBI221), pGA vectors (pGA492, pGAH), pNC (manufactured by Nissan Chemical Industries, Ltd.). In addition, virus vectors can also be mentioned. The terminator gene to be ligated includes 35S terminator gene and Nos terminator gene.

The expression system usable in the method of transforming prokaryote and eukaryote cells with the genes of the present invention include any system utilizing RNA, DNA sequences. It can be used to transform transiently or stably the selected host (bacteria, fungus, plant and animal cells) It includes any plasmid vectors, such as pUC, pBR, pBI, pGA, pNC derived vectors (for example pUC118, pBR322, pBI221 and pGAH). It also includes any viral DNA or RNA fragments derived from virus such as phage and retro-virus derived (TRBO, pEYK, LSNLsrc). Genes presented in the invention can be expressed by direct translation in case of RNA viral expression system, transcribed after in vivo recombination, downstream of promoter recognized by the host expression system (such as pLac, pVGB, pBAD, pPMA1, pGal4, pHXT7, pMet26, pCaMV-35S, pCMV, pSV40, pEM-7, pNos, pUBQ10, pDET3, or pRBCS.) or downstream of a promoter present in the expression system (vector or linear DNA). Promoters can be from synthetic, viral, prokaryote and eukaryote origins

The method of introducing the constructed expression vector into a plant includes an indirect introduction method and a direct introduction method. The indirect introduction includes, for example, a method using Agrobacterium. The direct introduction method includes, for example, an electroporation method, a particle gun method, a polyethylene glycol method, a microinjection method, a silicon carbide method etc.

The method of regenerating a plant individual from the transformed plant cells is not particularly limited, and may make use of techniques known in the art.

In another embodiment, the microcompartment proteins of the present invention can be produced by methods used conventionally for protein purification and isolation by a suitable combination of various kinds of column chromatography (e.g. gel filtration, ion-exchange), prepared by a chemical synthesis method using a peptide synthesizer (for example, peptide synthesizer 430A manufactured by Perkin Elmer Japan) or by a recombination method using a suitable host cell selected from prokaryotes and eukaryotes.

In another embodiment, an expression vector having any one of the nucleic acid sequences in SEQ ID NOS: 1 to 1268 and amplifiable in a desired host cells is used to transform bacteria, yeasts, insects or animal cells, and the transformed cells are cultured under suitable culture conditions, whereby a large amount of the protein can be obtained as a recombinant. Culture of the transformant can be carried out by general methods.

The method used in purifying the protein of the present invention from a culture mixture can be suitably selected from methods used usually in protein purification. That is, a proper method can be selected suitably from usually used methods such as salting-out, ultrafiltration, isoelectric precipitation, gel filtration, electrophoresis, ion-exchange chromatography, hydrophobic chromatography, various kinds of affinity chromatography such as antibody chromatography, chromatofocusing, adsorption chromatography and reverse phase chromatography, using a HPLC system etc. if necessary, and these techniques may be used in purification in a suitable order.

Further, the microcompartment proteins of the present invention can also be expressed as a fusion protein with another protein or a tag (for example, glutathione S transferase, protein A, hexahistidine tag, FLAG tag, etc.). The expressed fusion protein can be cleaved off with a suitable protease (for example, thrombin etc.), and preparation of the protein can be carried out more advantageously in some cases. Purification of the protein of the present invention may be carried out by using a suitable combination of general techniques familiar to those skilled in the art, and particularly upon expression of the protein in the form of a fusion protein, a purification method characteristic of the form is preferably adopted. Further, a method of obtaining the protein by using the recombinant DNA molecule in a cell-free synthesis method (J. Sambrook, et al.: Molecular Cloning 2nd ed. (1989)) is one of the methods for producing the protein by genetic engineering techniques.

A protein of the present invention can be prepared as it is, or in the form of a fusion protein with another protein, but the protein of the present invention can be changed into various forms without limitation to the fusion protein. For example, the processing of the protein by various techniques known to those skilled in the art, such as various chemical modifications of the protein, binding thereof to a polymer such as polyethylene glycol, and binding thereof to an insoluble carrier, may be conducted. The presence or absence of addition of sugar chains or a difference in the degree of addition of sugar chains can be recognized depending on the host used. The proteins in such cases are also construed to be under the concept of the present invention insofar as they function as proteins having microcompartment activity.

In one embodiment, an in-vitro transcription/translation system (e.g., Roche RTS 100 E. coli HY) can be used to produce cell-free microcompartments or expression products of the current invention.

In some embodiments, it is preferred that the microcompartments, comprising a Group of the microcompartment nucleic acids, proteins or polypeptides as selected from one of the 32 Groups, should provide an organism enhanced biomass production and CO₂ sequestration abilities, but however, be non-toxic or have low toxicity levels to humans, animals and plants or other organisms that are not the target.

In some embodiments, the expression cassette comprising the sequences of genes of one of the Groups of Table 1 are combined with a microcompartment protein from another Group of Table 1, i.e., any nucleotide sequence in SEQ ID NOS:1-1268, that encodes a microcompartment RNA or polypeptide can be selected and combined with any other. In another embodiment, a nucleotide sequence encoding a non-microcompartment protein, such as genes encoding plant RuBisCO, is combined with microcompartment expression cassettes.

The microcompartment proteins are preferably incorporated into a plant or microorganism to provide new or enhanced metabolic activity, and more often than not, to provide enhanced carbon fixation and sequestration activity in the plant or organism.

### Example 1

### Expression of Carboxysome (Components) from Synechocystis 6803 in Chlamydomonas

The expression of carboxysome (components) from Synechocystis 6803 in Chlamydomonas will provide an improvement of biomass production/CO₂ sequestration in Chlamydomonas by reduction of photorespiration using a CO₂ concentration “cage.” This will also provide groundwork for further engineering of Chlamydomonas and other algae with microcompartment-based catalysis.

Common to all strategies: (1) Gene synthesis for codon optimization for expression; (2) Systematic variation of the components included (CcaA, CcmM, RbcX, CcmN, etc); (3) Initially, transformation of cell wall mutant strain (displays high transformation efficiency) by glass-bead transformation or/and biolistic/gene-gun (electroporation as a last option due to plasmid size); (4) Antibiotic selection and PCR to check for complete integration, western blot analysis for shell protein expression, carboxysome formation and RuBisCO sequestration; (5) Confirmation of shell formation by EM; (6) Mating with wild type strain and carbonic anhydrase mutants (cia3 mutant and cia 6,7 if available) and screen for improve growth under low CO₂/O₂ ratio, preliminary test on solid media and extension to liquid media for CRII and CRIII strategies; (7) Option to apply directed evolution to optimize algal phenotype followed by resequencing.

Strategy CRI: Reconstitution of a carboxysome in Chlamydomonas cytosol: (1) Generation of vector for shell protein expression (+/− component enzymes) in Chlamydomonas cytosol. Co-expression of CcmK, L, and +/−N and +/−M and +/−CcaA and +/−RuBisCO large and small subunits from Synechocystis.

Strategy CRII: Reconstitution of a functional carboxysome that encapsulates Chlamydomonas RuBisCO: (1) Use of the vectors from CRI and insertion of chloroplast targeting signal peptide to target shell proteins +/− a subset of carboxysome interior components (N, M and CcaA); (2) Generation of a plasmid for chloroplast transformation to express directly shell proteins (CcmK, L) +/− component enzymes (N, M and CcaA) in Chlamydomonas chloroplast.

Strategy CrIII: Reconstitution of a complete cyanobacterial carboxysome into Chlamydomonas chloroplast: (1) Use of the vectors from CRII allowing the targeting of shell proteins, a subset of carboxysome interior components selected from CRI and CRII experiments and insertion of RuBisCO large and small subunit genes from Synechocystis; (2) Use of the vectors from CRII allowing chloroplast transformation for direct chloroplastic expression of shell proteins, subset of carboxysome interior components selected from CRI and CRII experiments and the RuBisCO large and small subunits from Synechocystis.

### Example 2

### C3-Plant Carboxysome Engineering

The present method also enables the improvement of biomass production in C3-plant by reduction of photorespiration/CO2 sequestration using a CO2 concentration “cage” from Cyanobacteria by reconstitution of carboxysome (components) from Synechocystis 6803 in C3-plants. Model species that can be used: Arabidopsis and Tobacco

Common to all strategies: (1) Gene synthesis for codon optimization for Arabidopsis/Tobacco expression; (2) Floral dipping for agro-transformation of Arabidopsis wild type and RuBisCO-mutants for nucleic integration of T-DNA carrying genes of interest; (3) Biolistic/gene-gun for chloroplastic transformation in tobacco; (4) Antibiotic selection and PCR check for complete integration, western blot analysis for shell protein expression, carboxysome formation and RuBisCO sequestration; (5) Confirmation of shell formation by EM; (6) Screen for improved growth under low CO₂/O₂ ratio.

Strategy AtI: Reconstitution of a carboxysome in Arabidopsis cytosol: Generation of T-DNA for shell protein expression (+/− component enzymes) in Arabidopsis cytosol. Co-expression of ccmK, L, and: Component enzymes: +/−N and +/−M and +/−CcaA and +/−RuBisCO large and small subunits from Synechocystis.

Strategy AtII: Reconstitution of a functional carboxysome that encapsulates Arabidopsis/Tobacco RuBisCO: (1) Use of the T-DNA from AtI and insertion of chloroplast targeting signal peptide to target shell proteins +/− a subset of carboxysome interior components (N, M and CcaA). Transformation of Arabidopsis plants; (2) Generation of a plasmid for chloroplastic transformation to directly express shell proteins (ccmK, L) +/− component enzymes (N, M and CcaA) in chloroplast. Chloroplast transformation in Tobacco (Tobacco as model system, because technique already well established).

Strategy AtIII: Reconstitution of a complete cyanobacterial carboxysome into Arabidopsis/Tobacco chloroplast: (1) Use of the T-DNA from AtII allowing the targeting of shell proteins, a subset of carboxysome interior components selected from AtI and AtII experiments and insertion of RuBisCO large and small subunit genes from Synechocystis. Transformation of Arabidopsis plants; (2) Use of the vectors from AtII allowing chloroplastic transformation for direct chloroplastic expression of shell proteins, subset of carboxysome interior components selected from AtI and AtII experiments and the RuBisCO large and small subunits from Synechocystis. Chloroplast transformation in Tobacco.

### Example 3

### Expression of Carboxysome (Components) from Synechocystis 6803 in Yeast

All microcompartment components can be expressed in yeast (wild type or mutant strains) after codon optimization. The advantage of codon optimization is that it will reduce the influence of translation efficiency and will facilitate optimizing protein ratio of each component of a desired micro-compartment. To generate micro-compartments in yeast, components need to be expressed with selected promoters and plasmids in order to obtain the right protein ratio for each component. Plasmids can be low or high copy replicative vectors (i.e. pRS series) or integrative (i.e.; YIplac series). Alternatively, plasmid can be replaced by a DNA fragment that will be integrated in the genome via targeted recombination to replace a host ORF by another one encoding for a component(s) of the micro-compartment. When plasmids are used, an expression cassette is usually required and consists of a gene(s) of interest inserted downstream of a selected promoter, which can be tunable (pMet26, pGal4) or constitutive (pPMA1, pADH, pPGK, pHHT7, or . . . ) to reach desired level of expression. Maintenance, selection or modification of a yeast is assisted by the use of antibiotic selection markers (kanamycin, Zeocin, hygromycin) or/and with auxotrophy markers (URA3, LEU2, HIS3, . . . ). For proteins that required to be expressed at equal ratio, chimera protein expression strategy can be used. It consists of the expression a large protein derived from the fusion of 2 or more proteins of interest. These proteins will be separated by a small protease recognition site, which will be cleaved in the host cell to produce the individual proteins. The production of micro-compartments in yeast will be achieved by expressing shell proteins with or without the internal components. For example, genes encoding for a carboxysome shell proteins such as pentamers (e.g. CsoS4A and CsoS4B) and (pseudo)hexamers (e.g. CcmK, CcmO, CcmP, CsoS1 and CsoS1D) will be expressed at high and low levels respectively and using a high copy plasmid and a genomic integration strategy respectively. This microcompartment could be used to isolate and to purify oxygen sensitive proteins (e.i. Pyruvate Formate-Lyase) or toxic proteins (e.i. RNase, ccdB protein). The sequestration of a desired protein this carboxysome can be achieved by the production of a chimera gene containing the sequences of a targeting peptide or the RubisCO subunits (e.g cbbS, cbbL), the protein of interest and a protease site (such as TEV) in between. The peptide or RubisCO subunit will allow the sequestration of the protein of interest into the micro-compartments and could be subsequently used for its purification (e.g. using an antibody targeted against the Ibbs). The protease will be used to cleave the RubisCO subunit or peptide from the protein of interest after purification.

In the case of the expression of a new enzymatic pathway that would be sequestered in a micro-compartment in yeast, the same strategy could be use to express the desired micro-compartment together with its native sequestered biosynthetic pathway.

### Example 4

### Expression of Carboxysome (Components) from Synechocystis 6803 in Bacteria

All carboxysome components can be expressed in bacteria (wild type or mutant strains) directly after codon optimization. The advantage of codon optimization is that it reduces the influence of translation efficiency and will facilitate obtaining the optimal protein ratio required to form a functional micro-compartment. The optimal expression levels for each component will be achieved using a combination of promoters that are, tunable (e.g. pVGB, pLAC and pBAD) or constitutive (pBLA, pPL, pSPC) and a combination of rbs sites. Selection of modified bacterial strain can be conduction under antibiotic selection (kanamycin, Zeocin, hygromycin) or/and with auxotrophy markers (uracil, leucine). For proteins that required to be expressed as equal level, they will be expressed together with the same promoter using the same rbs.

The production of microcompartments in E. coli can be achieved by expressing shell proteins with or without the internal microcompartment components. For example, the conversion of ethanolamine into ethanol and acetyl-CoA could be achieved by reconstituting a functional ethanolamine micro-compartment from Salmonella enterica. For this proposed transformation, a similar operon as in Salmonella (FIGS. 16A and 16B (Group 15, SEQ ID NOs: 773-814), FIG. 18 (Group 17, SEQ ID NOs: 1055-1098), FIG. 20A, 20B (Group 19, SEQ ID NOs: 903-936), or FIG. 22 (Group 21, SEQ ID NOs: 1099-1196) could be generated with known promoter and rbc and codon optimized sequences of genes encoding the microcompartment components. According to the level of expression that needs to be achieved for some of the components such as the hexameric shell proteins, a medium-high copy plasmid could be used (in contrast to the other components that would be carried in a low copy plasmid). These combinations of high-low copy plasmids, promoters and rbs sequences will allow one to achieve the correct expression ratio of each component. To reconstitute the ethanolamine microcompartment, a minimum of 9 proteins presumably are required: hexameric shell proteins (EutS, L and K; SEQ ID NOS:905,906; 933,934; 935,936), pentameric shell proteins (EutM and N; SEQ ID NOS:915,916; 917,918), AdoCbl-dependent ethanolamine ammonia-lyase complex (EutB and C; SEQ ID NOS:929,930; 931,932); aldehyde dehydrogenase (EutE; SEQ ID NOS:919,920) and alcohol dehydrogenase (EutG; SEQ ID NOS:923,924). Additional genes such as EutH (SEQ ID NOS: 925,926), could be expressed to together with these microcompartment genes to improve conversion efficiency. In such particular case, the transporter EutH would increase the import of ethanolamine into the cell.

Alternatively, the 9 proteins could be provided in a cassette where the genes are ordered substantially as their order appears in any of the Groups shown above. In one embodiment, the genes in the cassette are ordered substantially as their order appears in Group 19 as:. EutS (SEQ ID NOS:905, 906), EutM and N (SEQ ID NOS:915,916; 917,918); EutE (SEQ ID NOS:919,920); EutG (SEQ ID NOS:923,924); EutH (SEQ ID NOS: 925,926); EutB and C (SEQ ID NOS:929,930; 931,932); EutL and K; SEQ ID NOS: 933,934; 935,936).

### Example 5

### Enhanced Expression of Carboxysome (Components) with Other Activity in Bacteria

As described in Example 1, to reconstitute the carboxysome microcompartment, genes found in Group 12 and for example, genes encoding any of the following: PF00936 258aa, CcmN 304aa, Protein tyrosine phosphatase (COG0394), CcmM 672aa, PF03319 100aa [RGSA pore], PF00936 112aa [KIGS pore], PF00936 103aa [KIGS pore], the large (Pfam00016/02788) and small (Pfam00101) subunits of RuBisCO, the RuBisCO chaperone, RbcX (Pfam02341) and additional shell (Pfam00936) proteins, are expressed together with plant RuBisCO or RuBisCO activase from another cyanobacterium (e.g. Acaryochloris marina: locus tag AM1_{—}1781, Accession number YP001516116 to improve CO₂ fixation efficiency or enhance activity of the microcompartment.

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

TABLE 2
Table 2

| | Figure | SEQ ID | Representative | Potentially encapsulated | Organism |
|---|---|---|---|---|---|
| Group | Number(s) | Numbers | organism | reactions | phenotypes |
| 12 and 12A | 13A-D, other frags | 635-652, | Anabaena variabilis | Bicarbonate −> carbon | Aerobe |
| | in genome not | 653-668 | ATCC 29413, | dioxide −> glycerate 3- | |
| | shown | | Trichodesmium | phosphate | |
| | | | orythraeum IMS101 | | |
| 24A, 24B | 25A, 26A | 937-970, | Thiomicrospira | | |
| | | 971-944 | crunogena XCL-2, | | |
| | | | Prochlorococcus | | |
| 15, 19, 21 | 16A, 16B, | 773-814, | Salmonella typhimurium | Ethanolamine −> | Aerobe |
| | 20A, 20B, 22A | 905-938, | LT2 (Proteobacteria) | Acetaldehyde −> Acetyl- | |
| | | bacteroides | Clostridium | CoA | |
| | | | phytofermentans ISDg | | |
| | | | (Firmicutes), Alkaphilus | | |
| | | | metalliredigens, | | |
| | | | Bacteroides capillosus | | |
| | | | ATCC 29799 | | |
| 8, 18, | 9A, 9B, 18, | 387-436, | Salmonella typhimurium | 1,2-propanediol −> | Aerobe |
| | 19A, 19B | 861-902, | LT2, Desulfatibacillum | proprionaldehyde −> | |
| | | | alkenivorans AK-01, | propanol | |
| 4, 5, 6 | 5A, | 177-234, | Rhodopseudomonas | 1,2-propanediol −> | Generally |
| | 5B, 6A, 6B, 7A, 7B | 235-270, | palustris BisB18/E. coli | propionaldehyde −> | anaerobic; |
| | | 297-342 | CFT073/Shewanella | propanol | maybe |
| | | | putrefasciens CN-32 | | facultative |
| 2 | 3A, 3B | 69-98 | Ruminococcus obeum | Fuculose-1-phosphate −> | Anaerobe |
| | | | ATCC 29174 | lactaldehyde −> 1,2- | |
| | | | Clostridium | propanediol −> | |
| | | | phytofermentans ISDg | proprionaldehyde −> | |
| | | | | propanol | |
| 20 | 21A | 995-1054 | Clostridium kluyveri | Ethanol −> Acetaldehyde −> | Anaerobe; |
| | | | DSM 555 | Acetyl-CoA | Can grow on |
| | | | | | ethanol, |
| | | | | | acetate only |
| 9 | 10A, 10B | 535-550 | Blastopirellula marina | Fuculose-1-phosphate −> | Aerobe |
| | | | DSM 3645 | lactaldehyde −> ? | |
| 22 | 23A | 1197-1232 | Opitutus terrae PB90-1 | Fuculose-1-phosphate or | Obligate |
| | | | | rhamnulose-1-phosphate −> | anaerobe |
| | | | | lactaldehyde −> lactate | |
| 7, 13, 14, 16 | 8A, 8B, 14A, 15A, | 3434-386, | Clostridium | Unknown; Highest | Anaerobe |
| | 15B 17A, 17B | 669-714, | phytofermentans ISDg, | homology to glycerol | |
| | | 715-772, | E. coli UT189, | dehydratase, but not a GD | |
| | | 815-860 | Desulfotalea | | |
| | | | psychrophila LSv54, | | |
| | | | Alkaphilus | | |
| 21 | 22A | 1099-1196 | Bacteroides capillosus | N-acetyl-glutamylphosphate −> | Aerotolerant |
| | | | ATCC 29799 | N-acetylglutamate | anaerobe; |
| | | | | semialdehyde −> N- | pathogen |
| | | | | acetylomithine | |
| 1 | 2A, 2B | 1 to 20 | Mycobacterium | L-aspartate-4- | Aerobe, non |
| | | | smegmatis MC2 155 | semialdehyde or glutamate- | pathogenic |
| | | | | 5-semialdehyde based | |
| | | | | reactions | |
| 11 | 12A, 12B | 609-634 | Haliangium ochraceum | Homoserine <--> L- | Aerobe |
| | | | SMP-2 | aspartate-4-semialdehyde <−> ? | |
| 3 | 4A, 4B | 99-142 | Alkaliphilus | Hypoxanthine −> xanthine −> | Anaerobe |
| | | | metalliredigens QYMF | 5-ureido-4-imidazole | |
| | | | | carboxylate | |
| 10 | 11A | 561-608 | Methylibium | Unknown aldehyde | Aerobe |
| | | | petroleiphilum PM1- | metabolism | |
| | | | plasmid | | |
| 23 | 24A | 1233-1250 | Chloroherpeton | Unknown | Anaerobic, |
| | | | thalassium ATCC | | photoautotro- |
| | | | 35110 | | phic |
| 17 | 18 | 1055-1098 | Leptotrichia buccalis C- | | |
| | | | 1013-b | | |

| | Enzymes (proposed | Proposed Reason | |
|---|---|---|---|
| Group | from annotation) | for Encapsulation | Additional Notes |
| 12 and 12A | Carbonic anhydrase, | RuBisCO | |
| | RuBisCO | inefficiency, | |
| | | RuBisCO oxygen | |
| | | sensitivity, product | |
| 24A, 24B | | | |
| 15, 19, 21 | Ethanolamine ammonia | Oxygen sensitivity, | |
| | lyase (EutBC), | product | |
| | acetaldehyde | volatility/toxicity | |
| | dehydrogenase (EutE) | | |
| 8, 18, | 1,2-propanediol | Oxygen sensitivity, | |
| | dehydratase (PduCDE); | product | |
| | B12-dependent; | volatility/toxicity | |
| | propionaldehyde | | |
| | dehydrogenase (PduP) | | |
| 4, 5, 6 | Putative 1,2-propanediol | Oxygen sensitivity, | |
| | dehydratase, B12- | product | |
| | independent (GRE); | volatility/toxicity | |
| | propionaldehyde | | |
| | dehydrogenase (PduP) | | |
| 2 | Putative 1,2-propanediol | Product | A fusion of the B12- |
| | dehydratase, B12- | volatility/toxicity | independent 1,2- |
| | independent (GRE); | | propandiol dehydratase |
| | propionaldehyde | | and fuculose degradation |
| | dehydrogenase (PduP); | | pathways |
| | Fuculose-1-phosphate | | |
| | aldolase, lactaldehyde | | |
| | oxidoreductase | | |
| 20 | Aldehyde | Product | No nearby 03319 genes; |
| | dehydrogenase; alcohol | volatility/toxicity | Alcohol dehydrogenases |
| | dehydrogenase | | are probably |
| | | | encapsulated from |
| | | | experimental evidence |
| 9 | Fuculose-1-phosphate | Product | |
| | aldolase | volatility/toxicity | |
| 22 | Fuculose/rhamnulose-1- | Product | Nearly identical to the |
| | phosphate aldolase; | volatility/toxicity | enzymes found in |
| | aldehyde | | Planctomycetes but also |
| | dehydrogenase | | includes the rhamnulose |
| | | | degradation pathway |
| 7, 13, 14, 16 | Unknown glycyl radical | Oxygen sensitivity, | |
| | enzyme with homology | product | |
| | to glycerol dehydratase | volatility/toxicity | |
| 21 | N-acetyl- | Product | Contains entire glutamate - |
| | gammaglutamyl | volatility/toxicity | arginine conversion |
| | phosphate reductase, | | pathway; 2 00936 |
| | acetylornithine | | proteins, no nearby |
| | aminotransferase | | 03319s |
| 1 | Aldehyde | Product | |
| | dehydrogenase: | volatility/toxicity | |
| | aminotransferase type | | |
| | III | | |
| 11 | L-homoserine: NAD + | Product | |
| | oxidoreductase (not in | volatility/toxicity | |
| | BMC; in genome); | | |
| | dihydrodipicolinate | | |
| | synthase or other | | |
| | enzymes that function | | |
| | on L-aspartate-4- | | |
| | semialdehyde (not in | | |
| | BMC; in genome) | | |
| 3 | Xanthine | Xanthine toxicity | |
| | dehydrogenase; | | |
| | Xanthine hydrolase | | |
| 10 | PduP/EutE aldehyde | Product | |
| | dehydrogenase; putative | volatility/toxicity | |
| | glutathione dependent | | |
| | formaldeyde | | |
| | dehydrogenase | | |
| 23 | No readily apparent | Unknown | 2 pfam00936, 3 |
| | encapsulated enzymes | | pfam03319 scattered |
| | near 00936/03319 | | throughout genome |
| | proteins | | |
| 17 | | | |

What is claimed is:

1. An expression cassette comprising a cluster of microcompartment genes isolated from a bacteria, wherein the cluster comprising a set of microcompartment genes necessary for the expression of a microcompartment, wherein the microcompartment genes are selected from the gene sequences of SEQ ID NOS:1-1268.

2. A bacterial compartment expressed from an expression cassette of claim 1.

3. The expression cassette of claim 1 comprising groups selected from the following groups of sequences: SEQ ID NOS: 1-20, 21-44, 45-68, 69-98, 99-146, 147-176, 177-234, 235-270, 271-296, 297-342, 343-386, 387-436, 437-482, 483-534, 535-560, 561-608, 609-634, 635-652 and 1251-1260, 653-668 and 1261-1268, 669-714, 715-772, 773-814, 815-860, 1055-1098, 861-902, 903-936-, 937-970, 971-994, 995-1054, 1099-1196, 1197-1232, or 1233-1250.

4. A cell comprising in its genome at least one stably incorporated expression cassette, said expression cassette comprising a heterologous nucleotide sequence or groups of sequences of claim 1 operably linked to a promoter that drives expression in the cell.

5. The cell of claim 4 wherein the cell is bacterial, archeal, yeast, fungal or other prokaryotic or eukaryotic origin.

6. A plant comprising in its genome at least one stably incorporated expression cassette of claim 1.

7. The plant of claim 6 having new or enhanced carbon fixation activity as a result of the expression of said expression cassette.

8. A photosynthetic organism comprising in its genome at least one stably incorporated expression cassette of claim 1.

9. The photosynthetic organism of claim 6 having new or enhanced carbon fixation, biomass production or carbon dioxide sequestration activity as a result of the expression of said expression cassette.

10. An expression cassette comprising the expression cassette of claim 1 operably linked to a promoter that drives expression in a plant.

11. The expression cassette of claim 10 further comprising an operably linked polynucleotide encoding a signal peptide.

12. A plant comprising in its genome at least one stably incorporated expression cassette, said expression cassette comprising a heterologous nucleotide sequence of claim 10 operably linked to a promoter that drives expression in the plant.

13. The plant of claim 12, wherein said plant displays enhanced carbon fixation activity.

14. A transformed seed of the plant of claim 12.

15. A method for enhancing carbon fixation activity in an organism, said method comprising introducing into an organism at least one expression cassette operably linked to a promoter that drives expression in the organism, said expression cassette comprising a cluster of microcompartment genes isolated from a bacteria, wherein the cluster comprising a set microcompartment genes necessary for the expression of a microcompartment that has carbon fixation activity.

16. The method of claim 15, wherein the microcompartment genes are selected from the odd numbered gene sequences in the Sequence Listing.

17. The method of claim 15, wherein the cluster selected from the following groups of sequences: SEQ ID NOS: 1-20, 21-44, 45-68, 69-98, 99-146, 147-176, 177-234, 235-270, 271-296, 297-342, 343-386, 387-436, 437-482, 483-534, 535-560, 561-608, 609-634, 635-652 and 1251-1260, 653-668 and 1261-1268, 669-714, 715-772, 773-814, 815-860, 1055-1098, 861-902, 903-936-, 937-970, 971-994, 995-1054, 1099-1196, 1197-1232, or 1233-1250.

18. A bacterial microcompartment catalog comprising a total of 1286 sequences encoding bacterial microcompartments, the proteins of each of which can be inserted into a host organism capable of being expressed using an inducible expression system.

19. The expression cassette of claim 3 further comprising a gene encoding a microcompartment protein selected from another group from claim 3.

20. The expression cassette of claim 19, further comprising a nucleotide sequence encoding a non-microcompartment protein to improve CO₂ fixation efficiency or enhance activity of the microcompartment.
